(19) United States
(12) Patent Application Publication
(10) Pub. No.: US 2026/0023142 A1
(43) Pub. Date: Jan. 22, 2026

(54) Breast Imaging Systems and Methods

(71) Applicant: Yale University, New Haven, CT (US)

(72) Inventors: Robert Todd Constable, Madison, CT (US); Gigi Galiana, Guilford, CT (US)

(21) Appl. No.: 19/272,497

(22) Filed: Jul. 17, 2025

Related U.S. Application Data
(60) Provisional application No. 63/672,857, filed on Jul. 18, 2024.

(51) Int. Cl. G01R 33/44; A61B 5/00; A61B 5/055; G01R 33/3815; G01R 33/385; G01R 33/54

(52) U.S. Cl. CPC G01R 33/445; A61B 5/004; A61B 5/055; A61B 5/4312; G01R 33/3815; G01R 33/3852; G01R 33/543

(57) ABSTRACT

Described herein are compact breast MRI systems having (i) an open, field-cycling magnet configured to produce and cycle between a first and second non-uniform B₀ different from the first non-uniform B₀, and (ii) a second component configured to produce a nonlinear spatial encoding gradient. Importantly, the open, field-cycling magnet and the spatial encoding gradients are customized to a specific imaging application. In particular, the second component contains one or several nonlinear DC gradient coils for spatial encoding. It also contains one or several a radiofrequency coils geometrically configured to have a non-planar configuration. Lastly, both the RF coils and DC encoding gradients are tailored specifically to the first non-uniform B₀ magnetic field, the second non-uniform B₀ magnetic field, or both. Artificial intelligence models trained to read imaging data can be incorporated as a component of the MRI systems. Also described are methods of using the disclosed breast MRI systems.

### CROSS REFERENCE TO RELATED APPLICATIONS

This application claims priority to and benefit of U.S. Provisional Application No. 63/672,857 filed on Jul. 18, 2024, the disclosure of which is hereby incorporated herein by reference in its entirety.

### FIELD OF THE INVENTION

The invention is in the field of breast diagnostic imaging, particularly breast magnetic resonance imaging (MRI) systems and methods, and more preferably breast MRI systems and methods that use a combination of (i) a small, open, field-cycling magnet to cycle between nonhomogeneous B₀ fields, (ii) radiofrequency coils having a non-planar configuration for non-linear spatial encoding, and (iii) direct current (DC) gradients for non-linear spatial encoding.

### BACKGROUND OF THE INVENTION

In conventional magnetic resonance imaging (MRI) systems, a spatially uniform main magnetic field is needed in the imaging region because large field gradients dephase the signal before it can be acquired. This requirement of conventional MRI severely limits the design opportunities of the main magnet, and the dependence on linear gradient fields for spatial encoding also constrains the designs. In both cases, this typically leads to an MRI system with large, cylindrically shaped magnets surrounding an examination table where the patient is positioned. This closed design of the magnet is not suitable for imaging claustrophobic patients and does not allow easy medical interventions or imaging of obese individuals. Conventional MRI systems are also very expensive. The high manufacture and operational cost associated with building superconducting magnets with uniform magnet fields prevents widespread availability of MR imaging, especially for MR as an initial screening tool. Another challenge that conventional MRI systems encounter involves time, resource intensive exams, and need for experts to examine the results to arrive at a diagnosis. This requirement for human experts means that diagnosis highly depends on the experience of the analysis and often can be subjective. Accordingly, an unmet need exists to provide breast MRI systems and techniques that overcome the limitations of conventional MRI.

Therefore, it is an object of the invention to provide breast MRI systems that alleviate one or more of the limitations described above.

It is also an object of the invention to provide breast MRI systems with primary magnets, local gradient coils, and/or radiofrequency coils that are customizable and/or customized to breast anatomy.

### SUMMARY OF THE INVENTION

Described herein are compact MRI systems designed for breast magnetic resonance (MR) imaging, and are herein called compact breast MRI systems. The compact breast MRI systems have a first component containing an open, field-cycling magnet configured to produce and cycle between a first non-uniform B₀ and a second non-uniform B₀, that is different from the first non-uniform B₀ (B₀ denotes a magnetic field generated by a primary magnet in the MRI system). The compact breast MRI systems have a footprint of less than about 1 m². The compact breast MRI systems further contain a second component containing one or a plurality of second components operably linked to the first component and configured to produce one or a plurality of nonlinear spatial encoding gradients that span a three-dimensional volume space. Importantly, the open, field-cycling magnet and/or the spatial encoding gradients are customizable and/or customized to a breast magnetic resonance imaging. The customization can be hardware specific (e.g., geometry) and/or software specific (e.g., algorithms for image reconstruction). In some forms, the one or the plurality of second components contain one or more radiofrequency (RF) coils with non-planar configuration, a non-horizontal configuration, or both, geometrically configured to receive signal from spins precessing perpendicularly to the non-uniform B₀ magnetic field for the customized B₀ geometry described above. In some forms, the one or the plurality of second components contain DC gradients for non-linear spatial encoding, provided by gradient coils (e.g., local gradient coils such as x-, y-, and/or z-local gradient coils). Lastly, the non-linear spatial encoding gradients are tailored specifically to the first non-uniform B₀ magnetic field, the second non-uniform B₀ magnetic field, or both. Some advantages of these design features are that the open, field-cycling magnet, the radiofrequency coils, and/or the gradient coils are customized to provide improved sensitivity at greater tissue depths in breast being imaged and breast-specific spatial encoding gradients, such that the compact breast MRI system performs at a high signal-to-noise ratio for a given anatomical structure. In addition, nonuniform and nonlinear magnetic fields generally have fewer design constraints and are cheaper to build.

The ability to perform breast MRI using a nonuniform main magnetic field allows the compact breast MRI system to have an open magnet design, which has many practical advantages. The system is potentially inexpensive because of the relaxed construction requirements in building a magnet that does not need to generate a uniform main magnetic field or linear encoding gradients; the system can be more quiet than conventional MRI systems because in some forms, no gradient coils are required; and the open magnet design allows scanning of claustrophobic subjects. The open design also allows a compact breast MRI system having wider range of shapes and sizes (e.g., having a footprint of less than about 1 m²). For example, it allows a compact breast MRI system to be built into an examination table or wall. Other small devices could be designed for specific applications in imaging the breast. This allows physicians to perform preliminary scanning in the doctor's office or in the field, similar to how ultrasound imaging is used today, making it widely accessible in doctor's offices. Portable compact breast MRI systems are also envisioned. The technique also can be applied to MR based material analysis, MR spectroscopy, and solid material NMR. In some forms, as noted above, direct current (DC) gradients for non-linear spatial encoding can be generated using gradient coils (e.g., local gradient coils such as x-, y-, and/or z-local gradient coils). Lastly, in some forms the systems and methods incorporate one or more RF coils (non-planar, non-horizontal, or both) having a configuration that acquires signal from a breast in the context of alternative main magnet geometry. The RF geometry encompasses or achieves proximity to abreast, and the orientation of the RF is designed to capture the spatially varying orientation of precession arising from the non-uniform B₀ field.

These advantages are made possible by a combination of innovative techniques that liberate MRI from the requirement of a uniform main magnetic field. In contrast with conventional MRI, the compact breast MRI systems described herein can provide high quality images using a nonuniform main magnetic field. This is facilitated by using an electromagnet to generate and cycle a nonuniform main magnetic field between a high field for spin polarization and a lower field for slice selection and MR signal readout; using multicoil arrays for spin excitation and receiving MR signals; and using non-linear DC gradients optionally in combination with RF spin-manipulation, for spatial encoding. The provisional of high quality images can also be facilitated by using an electromagnet to generate and cycle a nonuniform main magnetic field between a high field for spin polarization and a lower field for slice selection and MR signal readout; using multicoil arrays for spin excitation, receiving MR signals, and non-linear spatial encoding, where the multicoil arrays, preferably in a non-planar and/or non-horizontal configuration, produce a spatial encoding gradient that spans a three-dimensional volume space. Preferably, the image is reconstructed using reconstruction techniques and parallel imaging. To address the need for expert, and potential subject assessments of imaging data, artificial intelligence models (machine learning and/or deep learning algorithms) can be trained to assist in reading the imaging data, tested, and incorporated as a component of the compact breast MRI systems.

Also described are methods of using the disclosed compact breast MRI systems. An electromagnet is used to generate a spatially nonuniform magnetic field within an imaging region. By controlling current through the electromagnet, the nonuniform magnetic field is repeatedly cycled between a first strength for polarizing spins and a second strength, lower than the first strength, for spatial encoding and readout. Using one or more RF coils with appropriate configuration, excitation pulses are generated at a frequency that selects a non-planar isofield slice for imaging. The RF coils are also used to generate refocusing pulses for imaging and optionally to generate spatial encoding pulses. An alternative to using the RF coils to generate spatial encoding pulses, is the use of non-linear DC gradients obtained from gradient coils (e.g., local gradient coils such as x-, y-, and/or z-local gradient coils). Magnetic resonance signals originating from the selected non-planar isofield slice of the nonuniform magnetic field in the imaging region are detected using the RF coils in receive mode. The receive mode can be parallel (e.g., multi-channel) or single channel. Breast MRI images are reconstructed from the received magnetic resonance signals with spatial encoding provided by either RF spatial encoding, DC nonlinear gradient encoding, or their combination.

The nonuniform magnetic field can have a large spatial variation exceeding 5 ppm, 10 ppm, 20 ppm, 30 ppm, 40 ppm, 50 ppm, 60 ppm, 70 ppm, 80 ppm, 90 ppm, or 100 ppm within the imaging region, such as between 5 ppm and 500 ppm, preferably between 100 ppm and 500 ppm.

The electromagnet may be an open-geometry electromagnet that extends around the imaging region by no more than 270 degrees.

The first strength for polarizing spins can be at least 0.2 T (e.g., from 0.2 T to 1.0 T) and the second strength for spatial encoding and readout can be at most 0.1 T (e.g., from 10 mT to 0.1 T). In some forms, the first strength can be at least 0.6 T (e.g., 0.6 T to 1.0 T) and the second strength for spatial encoding and readout can be at most 0.1 T (e.g., 24 mT to 0.1 T).

In some forms, the RF coils contain a phased array of coils. The spatial encoding is nonlinear spatial encoding. The spatial encoding can be generated via the Bloch-Siegert shift or other spatial encoding pulses. The refocusing pulses generated using RF coils may include 180-degree RF pulses inserted to refocus the effects of the residual static gradient fields.

In some forms, the RF coils may contain first and second subsets of coils with no common coils, and spatial encoding can be generated using the first subset of the RF coils, while selecting a non-planar isofield slice and detecting magnetic resonance signals using the RF coils in parallel receive mode uses the second subset of the RF coils.

In some forms, the RF coils may contain first, second, and third subsets of coils with no common coils, and the first subset is used to generate spatial encoding, the second subset is used to select a non-planar isofield slice, and the third subset is used in parallel receive mode to detect magnetic resonance signals.

In some forms, spatial encoding can be performed with gradient coils that alter the local static (DC) magnetic field. Spatial encoding can be achieved solely by these gradients, which are non-linear. Spatial encoding by DC gradients can also be complemented with either RF encoding (e.g., Bloch-Siegert), the spatial encoding associated with parallel receive mode RF, or both. These gradients can also be used for spatially selective excitation.

In some forms, reconstructing breast MRI images from received magnetic resonance signals (e.g., multichannel imaging or single channel imaging) uses algebraic reconstruction. Preferably, the breast image reconstruction does not use standard Fourier-based image reconstruction because it cannot accommodate the nonlinear spatial encoding fields that are used for spatial localization.

### BRIEF DESCRIPTION OF THE DRAWINGS

FIGS. 1A-1E: a) The design of a two-element field-cycling electromagnet. Winding for a single coil element (a), produces a nonuniform polarizing B0 field ranging from 0.6 T closest to the coil to 0.2 T at approximately 10 cm depth (measured vertically from the plane). B0-field direction is primarily L/R. Current is adjusted to yield high B0 field for polarizing the spins (maximizing signal), and a low field (24 mT, 1 MHz) during readout (minimizing non-uniform B0 effects). Such a magnet design can be used for breast MRI with the patient in the prone position as the field lines follow the contour of the chest wall and the maximum signal is obtained in the breasts. The insert plot shows long T2-decay from 800 echoes following excitation. Most conventional pulse sequences are available as are additional contrast mechanisms through field-cycling. Signal intensity is determined by the polarizing field.

FIGS. 2A-2C. a) NuBo magnet—showing that the B0 field has a slice selective gradient in the AP direction. The direct current (DC) gradients in S/I (b) and L/R (c) are also shown for the left-breast (mirror images of these are made for the right-breast. With these local gradient coils and tight geometry, we can obtain sufficient spatial encoding for high-resolution (1 mm in-plane) imaging with only 10 A of current. The asymmetry in (c) is there to facilitate imaging into the chest wall and include the axillary lymph nodes.

FIGS. 3A-3D. The preliminary EM modelling of a close-fitting Tx/Rx solenoid coil. (A)) The prototype schematic of the solenoid Tx/Rx coil designed based on the low frequency MOSFET switch75. (B) The solenoid geometry (top/bottom diameter of 17 cm/12 cm, with height of 10 cm), and the breast/chest wall phantom geometry (diameter of 14 cm and height of 10 cm for breast with cup size 34DD, and a slab of 4 cm for chest wall). (C) The simulated flip angle map in the sagittal plane based on solenoid geometry (scaled so that the center of the breast is 90° FA). Within the breast, the mean FA is 89.6° and relative standard deviation (RSD) is 4.77%, suggesting a homogenous76 Tx pattern. (D) The simulated flip angle map in the coronal view (scaled such that the center of the breast is 90° FA).

FIG. 4: Wiring diagram for an alternate solenoid for breast imaging (upper left panel). This magnet produces a polarizing magnetic field in the range of 0.4 to ˜1 Tesla in peak regions. This magnet employs field cycling in a manner that the signal is equivalent to that from a 0.4-1.0 T magnet, whereas the actual imaging experiment is performed at 24 mT or 1 MHz. The low field readout allows nonuniform B0 by minimizing the effects of the field inhomogeneities. The 1 MHz plane is scanned through the breast and chest wall by adjusting the polarization field just like a slice-select gradient. The slices obtained are slightly non-planar (bottom right panel) but this is acceptable for diagnosis (and it could be reformatted into planar slices but in general this is not necessary).

FIG. 5: Modified Golay coils to generate spatial encoding gradients with only 25 amps of current. The orthogonal gradient is generated simply with another coil of this same design, rotated 900 about the vertical axis.

FIGS. 6A-6D. The preliminary EM modelling of the close-fitting Tx/Rx Saddle coil for breast imaging. (A) The prototype schematic of the solenoid Tx/Rx coil designed based on the low frequency MOSFET switch75. (B) A picture of the saddle geometry (spanning 120° for each side of saddle). (C) The simulated flip angle map in Sagittal plane based on the saddle geometry. The map is scaled such that the center of the breast has a 90° FA. (D) The simulated flip angle map in Coronal plane based on the saddle geometry, again scaled such that the center of the breast has a 90° FA.

FIG. 7: B0 polarization reversal for T1 nulling. The rampable B0 can also be used to generate contrast. After reversal the field is on for time t-until either fat or water goes through zero, at which point the B0 is cycled to low field for imaging. Middle panel shows the recovery curve for water as a function of polarization reversal time τ−. Right panel shows experimental nulling of oil or water dependent upon τ−. With τ−=2.5 ms the oil is nulled while the water signal remains high (top row panel 3), whereas the opposite is true with τ−=800 ms, wherein the oil signal is high, and the water is nulled.

FIG. 8: Diffusion weighting with B0 magnet. Because of the nonuniformity of the B0 magnet, it can also be used to generate diffusion contrast. Strong diffusion encoding applied after the polarization period can be used to generate diffusion contrast. Very high b-values can be obtained with the intrinsic B0 field.

FIGS. 9A-9C: Field cycling with the NuBo system shows the command signal used to drive the electromagnet (blue lines) and the current through the magnet (red). The boxes in panel (a) and expanded in panels (b) and (c) and highlight the responses (and stabilization time (red) following the inputs (blue) for both rising amplitude (b) and falling amplitude (c). The stepped trajectory shown in (a) illustrates how slices can be selected as the 1 MHz isofrequency band is scanned through the object to be imaged. The settling time for the increasing field is of the order of 0.8 ms, and for the falling field it is of the order of 1.1 ms. After the field is lowered to 1 MHz in the plane of interest, the image encoding pulse sequence is applied to collect an image of that slice.

FIG. 10: Block diagram of the work distribution of the system. The controller can interface with the computer as well as each of the peripheral boards. The number of Tx/Rx boards is configurable.

FIGS. 11A and 11B. A pyramid phantom (a) composed of hollow pyramids and filled with doped water. The pyramids allow calibration of slice profiles, and image sharpness throughout the FOV. (b) A contrast detail phantom to measure contrast and resolution. Variants of these phantoms that custom fit inside the breast coils can be built to characterize system performance.

FIG. 12: Alternate magnet design for breast imaging. The two-element coil loops are shown in the top right panel (similar to what is shown in FIGS. 1A-1D) and the generated polarizing field including in the chest wall is shown in the top right. A field map through the breast and chest wall is shown bottom left, along with the slice shapes (slightly nonplanar) shown on bottom right. The color bar in the top right applies to all the panels.

FIGS. 13A-13F. The preliminary EM modelling of the 4-channel “Hammock” ladder-network Rx Array coil. (A) A picture of the modelled geometry of the 4ch breast array coil. (B.1-B.4) Simulated individual B1 pattern from the Rx coil element #1 to #4 in Coronal plane. (B) Combined array B1 pattern from each element in Coronal plane using sum of square. (C.1-B.4) Simulated individual B1 pattern from the Rx coil element #1 to #4 in Sagittal plane. (C) Combined array B1 pattern from each element in Sagittal plane using sum of square.

FIGS. 14A-14D. The preliminary EM modelling of the 4-channel Circular Rx Array coil. (A) A picture of the modelled geometry of the 4ch breast array coil. (B) The prototype schematic of the array coil designed based on the low frequency MOSFET switch73 (C) Simulated individual B1 pattern from the Rx coil element #1 to #4 in Coronal plane. (C) Combined array B1 pattern from each element in Coronal plane using sum of square.

### DETAILED DESCRIPTION OF THE INVENTION

### I. Definitions

“Nonuniform,” as relates to a magnetic field, refers to a magnetic a field with more than about 100 ppm variation with respect to changes in spatial location in an imaging region. The field gradient of a nonuniform field could be linear or nonlinear. The orientation of the magnetic field vector can also vary considerably with respect to spatial location in an imaging region.

“Operably linked” refers to the connection of at least two components in an MRI system via technology including, but not limited to, integrated circuits, electrical cables, ethernet, internet, intranet, Bluetooth, near field communication, WiFi, or a combination thereof.

“Open,” as relates to the geometry of a magnet, refers to a magnet that does not completely surround an imaging region. As an example, the magnet surrounds the center of the region by at most 270 degrees.

### II. Magnetic Resonance Imaging Systems and Methods

Disclosed herein are breast MRI systems, preferably compact breast MRI systems, designed from breast MR imaging. The breast MRI systems contain a first component containing an open, field-cycling magnet configured to produce a first non-uniform B₀ magnetic field and a second non-uniform B₀ magnetic field having a field strength different from that of the first non-uniform B₀ magnetic field, and one or a plurality of second components operably linked to the first component and configured to produce one or a plurality of nonlinear spatial encoding gradients. Nonlinear encoding, as relates to nonlinear spatial encoding encompasses both highly nonlinear as well as modest deviations from linearity. Preferably, the non-linear spatial encoding gradients in the breast MRI systems described herein span a three-dimensional volume space, whether generated using RF coils in a non-planar configuration, a non-horizontal configuration, or both; using gradient coils (e.g., local gradient coils such as x-, y-, and/or z-local gradient coils) using DC; or a combination thereof. Preferably, the breast MRI system is a compact breast MRI system (e.g., having a footprint less than about 1 m²) and suitable for operation in multi-purpose spaces or mixed-use rooms.

In some forms, the first non-uniform B₀ magnetic field has a field strength greater than that of the second non-uniform B₀ magnetic field and/or is configured to generate a slice-selective gradient. In some forms, one or plurality of second components contain: (i) one or more radiofrequency coils; (ii) one or more nonlinear DC gradient coils; or (iii) both (i) and (ii); capable of being configured to produce one or plurality of nonlinear spatial encoding gradients. In some forms, one or plurality of second components contain: (i) one or more radiofrequency coils; (ii) one or more nonlinear gradient coils; or (iii) both (i) and (ii); capable of being tailored specifically to the first non-uniform B₀ magnetic field, the second non-uniform B₀ magnetic field, or both. The alternative geometry generates a B0 magnetic field that can be non-uniform in both magnitude and orientation. RF must be tailored with sensitivity that complements the field direction at each location.

In some forms, one or a plurality of second components contain one or more radiofrequency coils, wherein the one or more radiofrequency coils have a non-planar configuration, a non-horizontal configuration, or both.

In some forms, one or a plurality of second components contain DC gradients for non-linear spatial encoding, preferably provided by gradient coils (e.g., local gradient coils such as x-, y-, and/or z-local gradient coils). In these forms, spatial encoding can be performed with gradient coils that alter the local static (DC) magnetic field. Spatial encoding can be achieved solely by these gradients, which are non-linear. Spatial encoding by DC gradients can also be complemented with either RF encoding (e.g., Bloch-Siegert), the spatial encoding associated with parallel receive mode RF, or both. These gradients can also be used for spatially selective excitation.

The disclosed breast MRI systems facilitate imaging using a main magnet with a highly nonuniform field operably linked to components configured to produce nonlinear spatial encoding gradients. This is made possible using techniques such as nonlinear MR imaging, field cycling MRI, RF based nonlinear spatial encoding, and imaging (e.g., multichannel imaging or single-channel imaging) with algebraic reconstruction. Preferably, the breast MRI systems are compact breast MRI systems.

Preferably, the breast MRI system (preferably compact breast MRI system) includes a computer that is connected to one or several electromagnet power supplies, to an RF transmitter, and to an RF receiver. One power supply is connected to an open geometry electromagnet that generates a nonuniform magnetic field in an imaging region. The nonuniform magnetic field at least includes a nonuniform polarizing magnetic field. The electromagnet preferably contains one or more resistive coil elements. An open geometry in the present description means that the magnet does not completely surround the imaging region, e.g., it surrounds the center of the region by at most 270 degrees. The RF transmitter and RF receiver are connected to one or more arrays of RF coils positioned near the imaging region. In some forms, the open, field-cycling magnet; the spatial encoding gradients; or a combination thereof, are customizable and/or customized to a specific imaging application. In preferred forms, the open, field-cycling magnet and the spatial encoding gradients are customizable and/or customized to a specific imaging application.

More details of the disclosed breast MRI systems (preferably compact breast MRI systems) and methods are provided in the ensuing sections.

Throughout this disclosure, preferred breast MRI systems, are compact breast MRI systems.

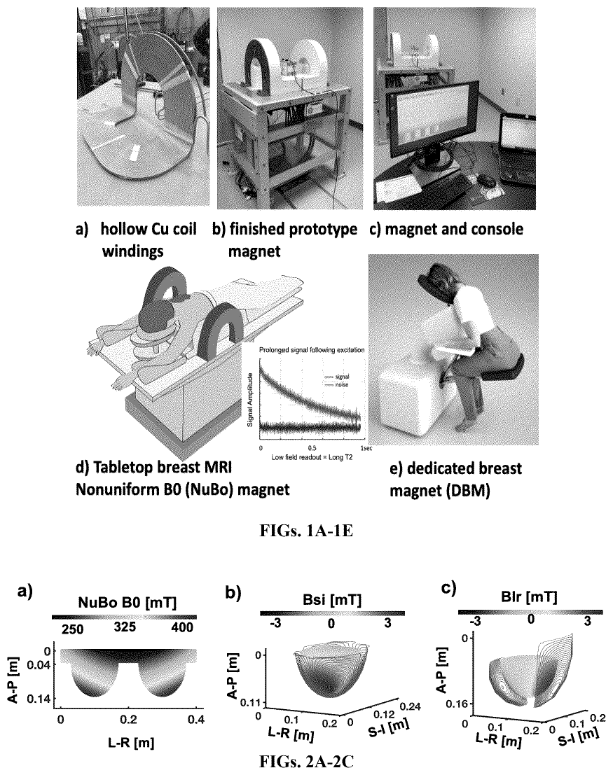

An important feature of the disclosed approach is the embracing of a nonuniform B₀-field in the magnet design. By eliminating the uniform B₀ constraint-which is the hallmark of most MRI systems-we free up the design possibilities and can tailor the magnet to the specific clinical application of interest. Currently most MRI systems use a cylindrical magnet, and only the RF coils are tailored to the specific application or anatomy. In the disclosed design the primary magnet, the RF and the spatial encoding gradients (DC spatial encoding gradient and/or spatial encoding gradients from the RF coils), are customized to breast imaging. In conventional MR imaging magnets with significant B0 inhomogeneities cannot be used. Our approach allows high resolution imaging with a highly nonuniform B₀ magnet because we use a technique called field cycling. Field cycling allows us to polarize the spins at high field (thereby generating strong signal) and image at low field (thereby minimizing the effect of the inhomogeneous field) these innovations are described below. Reducing the B₀ field after spin polarization has two other advantages: 1) it reduces the RF power needed to manipulate spins (hence low-cost amplifiers can be used, and it also has the benefit of low specific absorption rate (SAR); and 2) it yields long T2s making very long echo trains feasible. We have designed and built such a system and proven it's feasibility for imaging. The original design, shown in FIGS. 1A-1D was aimed at generic body imaging with the goal of building an MRI system into a patient examination table. Spatial encoding in this initial prototype has been performed solely with RF^{1,2} and parallel receiver coils. We have shown that this nonuniform B0 (NuBo) system works as intended. However, features can be implemented to improve image quality for breast MR imaging. To achieve this breast-specific feature, RF coils can be included to increase the signal-to-noise ratio (SNR) (moving away from the planar RF arrays). DC spatial encoding gradients can also be used to increase spatial resolution. Spatial encoding can be wholly performed entirely by the aforementioned gradients, a combination of spatial encoding gradients and receive coils (e.g., parallel receive coils), a combination of spatial encoding gradients and RF encoding, or a combination of all three.

FIGS. 1A-1D illustrate a design we have built (the white, U-shaped magnet in the middle of the top row in the figure). The angular coil loops are curved up to enhance the field at depth (above the tabletop). Each coil element is composed of hollow copper wires (with deionized water for efficient cooling) with a maximum current of 275 Amps. Data disclosed herein show experimental proof that we can polarize spins at high field and readout signal at low field. It has also been observed that prepolarization increases SNR. In this operation, strong signals are obtained, with echoes recorded for up to 1 second after an excitation pulse. The low field readout allows short 180° RF pulses (for turbo-spin echo acquisitions) and yields very long T2s (we can collect 800 echoes in less than 1 second). In addition to enhancing this NuBo system by adding breast-specific gradients and RF, field-cycling and inhomogeneous B0 magnets in a geometry completely redesigned for breast-specific imaging can be developed. In a non-limiting example, a dedicated breast-magnet (DBM) can be developed. These redesigns can be compared directly with the NuBo before finalizing a design and evaluating the breast MRI system in clinical applications. After determining a design with better performance compared to the NuBo (e.g., between NuBo and DBM systems), the design criteria and parameters of the prototype can be finalized for subsequent assessment of any safety concerns.

The breast MRI systems can be housed in clinical settings, and performance assessed in patient studies as well as assess ease of use in a clinical screening environment. FIG. 1E illustrates the open magnet for a non-limiting breast-specific application.

A goal of moving MRI out of the large hospital settings and into a community environment also includes simplifying the imaging process. While conventional MRI scanners have 100s, if not 1000s, of free parameters that can be chosen for each scan, the breast MRI systems can be designed with fixed protocols having a simple operation. The technologist would simply put the subject in position and execute instructions on a device (e.g., a hand-held device such as a tablet (e.g., iPad), mobile phone (e.g., a smart phone) or other hand-held devices) for scan 1, 2, 3, etc. Such a design allows for ease of use of breast-specific MRI in a community setting and even in diagnostic service centers such as Quest Diagnostics or CVS. New contrast mechanisms are available with field cycling as is diffusion contrast potentially leading to very short scan times. The model includes remote radiology reading of the scans through a radiology service. By ensuring identical scan protocols at all sites, this is a well-adapted scenario for artificial intelligence (AI) approaches to assisted reading³, and a very large data base could be acquired quickly—all with the exact same protocol for breast-specific MRI.

There are multiple innovative aspects of the disclosed breast MRI systems and these range from the hardware to software, to how the data is ultimately used (AI assisted radiology). These developments are briefly reviewed in the following paragraphs.

### A. Magnet Design (Field-Cycling)

Using an electromagnet combined with field-cycling provides the freedom to design open breast MRI systems customized to specific imaging applications as in the disclosed breast-specific MRI system.

### A.1 Nonuniform B0 Magnet (NuBo)

We have already designed and built an innovative, open, field-cycling, electromagnet, for body imaging and proven the feasibility of this technology. Field-cycling provides two benefits in this context. First, we can use a relatively high field for spin polarization thereby avoiding the very low SNR problems of low-field systems. The second benefit is that by lowering the B0-field during the imaging part of the sequence (after a pre-polarization phase) we avoid the problems associated with field inhomogeneities, and this provides flexible magnet design opportunities allowing new open designs to be developed-such as those disclosed herein.

Our existing magnet (a nonuniform B0 system we refer to as NuBo) was designed to be built into a patient exam table for general body imaging applications in doctor's offices (FIGS. 1A-1D). This can be used for breast imaging in the prone position, in that the highest polarizing fields encompass the breast and follow the contours of the chest wall which should provide outstanding signal-to-noise, contrast and resolution. The NuBo magnet was built for generic applications, and as such needs improvements for improved breast-specific MRI. To enhance this system for breast imaging two new developments are described herein: 1) The design of breast specific transmit and receive RF coils, and 2) breast-specific spatial encoding gradients. These developments can provide high-resolution, high-SNR breast MRI.

The existing NuBo system has a 3×3 flat panel set of array coils and relies on the Bloch-Siegert shift for spatial encoding. The geometry of the breast imaging application, however, makes it possible to develop breast-imaging specific spatial encoding gradients (and RF) that allow for much higher spatial resolution and signal-to-noise ratio than exists with the current setup. NuBo's polarization field is oriented in the left-right direction. This polarization field creates a slice-selective magnetic field that varies in the anterior-posterior direction (FIG. 2A). To achieve spatial encoding, in a traditional Fourier encoding approach, separate gradients that achieve magnetic field variation in the left-right and superior-inferior directions can be developed.

Our target field of vision (FOV) for each breast, is 14 cm×14 cm (in-plane) by 10 cm depth (fits an E sized breast cup)⁵⁴ with a 2 ms readout gradient and an in-plane resolution between 0.5-1 mm. The two breasts can be imaged simultaneously—each breast using a separate pair of Gsi, Glr spatial encoding gradients, and RF coils. The breasts can be closely cupped by transmit (Tx) and receive (Rx) RF coils. The Gsi gradient coils (LR: 16 cm, SI: 17 cm, AP: 11 cm) can cup the RF coils and the breast (FIG. 2b). Next, the Glr gradient coils (LR: 18 cm, SI: 17 cm, AP: 12.5 cm) can cup the Gsi, RF coils, and the breast (FIG. 2c). With these gradient dimensions, the tip of the breast can be 3.5 cm away from the NuBo magnet surface where polarization is strongest. The coil windings of the gradients were redesigned to achieve strong linearly varying gradient fields in the LR and SI directions using gradient coil generating software: CoilGen⁵⁵. The gradients must be affordable to keep the cost of the device low, and these gradients require less than 25 Amps current which can be obtained with ˜$3500 amplifiers⁵⁶.

### Specific, Non-Limiting Steps, Include:

Wind gradient coils (shaped as in FIGS. 2A and 2C).
Embed gradient coils in epoxy.
Connect coils to gradient cooling system.
Test gradient coil heating-repeat gradient system running at maximum power for readout time multiple times, measure temperature changes of coils, and temperature changes at breast FOV.
Test gradient stability-repeat gradient system running at maximum power for readout time multiple times, measure stability of gradient field at multiple positions within the FOV.
Test duty cycle repeat gradient system running at maximum power for 2 ms readout multiple times.

| Acceptance criteria: | Gsi | | Glr | |
|---|---|---|---|---|
| Gradient strength | minimum: 25 mT/m, | | minimum: 25 mT/m, | |
| | target | | target | |
| | maximum: 40 mT/m | | maximum: 40 mT/m | |
| Rise time | minimum 0.5 ms, | | minimum 0.5 ms, | |
| | target | | target | |
| | maximum: 0.8 ms | | maximum: 0.8 ms | |
| Slew rate | 50 | T/m/s | 50 | T/m/s |
| Gradient duty cycle | 2 | ms | 2 | ms |
| Readout time | 2 | ms | 2 | ms |
| Points | 256 | | 256 | |
| Resolution | ~0.5 | mm | ~0.5 | mm |
| Heating during | <1° | C. | <1° | C. |
| readout | | | | |

### Transmit (Tx) and Receive (Rx) RF Coils for NuBo

The planar array we currently have on the NuBo system requires redesigning for improved breast MRI. Therefore, we can replace this with volume coils that developed to encompass the breast. Many geometries of dedicated RF coils have been used in breast MRI studies. In the early stage of MRI development, simple close-fitting volume breast coils, such as Helmholtz pair⁵⁷ and conical solenoid⁵⁸, were used in preliminary studies. Later on, relatively more sophisticated volume coil designs, such as crossed-oriented loop pair⁵⁹ and Helmholtz/Saddle pair⁶⁰, incorporating quadrature modes. After the introduction of phased array coil in 1990⁶¹, different array designs dedicated to improve breast MRI's sensitivity while reducing scan time have been reported, such as soccer-ball array^{62,63}, ladder-network array⁶³, and circular loop array⁶⁴. Many folds of SNR improvement can be found in these studies. With the help of flexible coil technology developments, new breast array design of flexible PCB array coil⁶⁵, wearable-vest-type breast coils^{66,67} have been proposed for improving the coil's fill factor and comfortability. In low-field MRI there are advantages in terms of RF safety (less power is needed)⁶⁸. Most low-field breast MRI studies have used volume coils, such as quadrature saddle pair⁶⁹, bilateral solenoid⁷⁰, or optimized conical solenoid⁷¹. Attempts to implement array coils on low field scanners have been reported⁷² (although in that report each coil element was acquired individually to mimic parallel performance), but there are challenges because of the coil's form factor⁷³ with the geometry of the breast. In a non-limiting example, the disclosed breast MRI systems can initially include a simple but effective close-fitting volumetric solenoid coil. Other coil configurations can also be included, such as parallel Rx array coil, in “Hammock” ladder-network geometry⁶⁴ to facilitate parallel imaging for faster acquisitions.

It is contemplated that the developed RF coils yield high sensitivity. Our coil design choices rely on coil geometries that have been long-tested in conventional MRI but here, customized for low field nonuniform MRI, to achieve optimal SNR/homogeneity performance in analog to the conventional field strength using a transmit-only/receive-only (TORO) configuration⁷⁴. The designs also account for nonstandard and spatially varying orientation of the magnetic field, which leads to a need to detect precession of spins in nonstandard and spatially varying planes.

### New Volumetric Tx/Rx Solenoid Coil

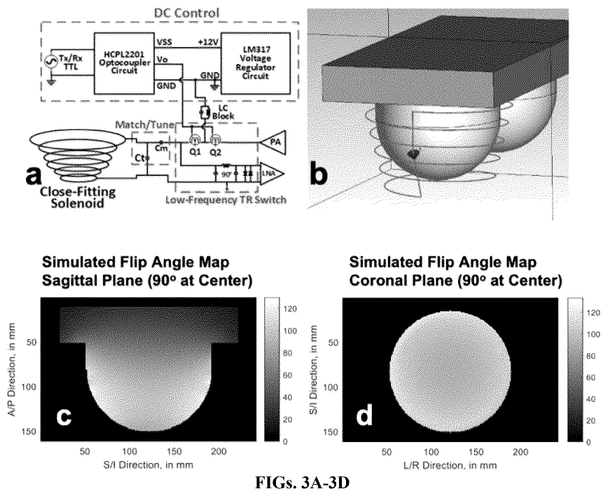

For breast anatomy, a solenoid coil design yields excellent homogeneity over different breast sizes⁵⁸ and field-of-views⁷¹. We have improved this design using EM Simulation and Optimization. The solenoid structure was modelled using CST (Dassault Systems Deutschland GmbH), following previously reported optimization strategies^{58,71}. The design and initial modelling results demonstrate a homogeneous transmit pattern, and are shown in FIGS. 3A-3D.

### Specific, Non-Limiting Steps, Include:

Development and Bench Testing. The improved solenoid can be built and tested on the bench, including B1+ mapping to verify with the simulation, using the bench mapping system developed in the lab.
Scanner Testing. The solenoid will be tested and used on scanner as a Tx/Rx coil, measuring B1 amplitude and uniformity as well as flip angle per unit input power.
A custom-shaped volumetric Tx/Rx solenoid RF coil for breast imaging on the NuBo system (in the case of a breast-specific MRI application) can be developed. A more advanced parallel Rx version can be constructed in Phase 2 if we move ahead with the NuBo system.

### Integration to Complete NuBo

The rest of the NuBo system is built and can easily accommodate these spatial encoding gradients and new RF coil designs. Similarly, we have implemented active electromagnet interference (EMI) sensors to detect noise and subtract this from the MRI signal such that imaging can be performed in the absence of an RF shielded room (making siting much easier than for conventional MRI systems). We can use a suitable console, such as our existing Tec-Mag console, for this work.

### A.2 Dedicated Breast Magnet (DBM)

The NuBo system was built as a general MRI system for body imaging and designed to be built into an exam table with capabilities of performing cardiac, spine, and liver imaging, in addition to breast imaging. The design criteria to produce a breast-specific MRI device are different, however. In a non-limiting example, here we can build and test a dedicated breast magnet which should have some advantages by better matching the geometry to the application. An innovative new design is described based on the same field-cycling principles. This system generates a higher polarization field than NuBo (and hence more signal) because of the tailored shape of the magnet. Tests can be performed to determine whether this dedicated-breast-magnet can be built with a smaller footprint, lower cost, and yet provide a higher, more uniform polarizing field for maximizing signal compared to the NuBo system with breast-specific gradient and RF hardware.

### Build DBM Magnet

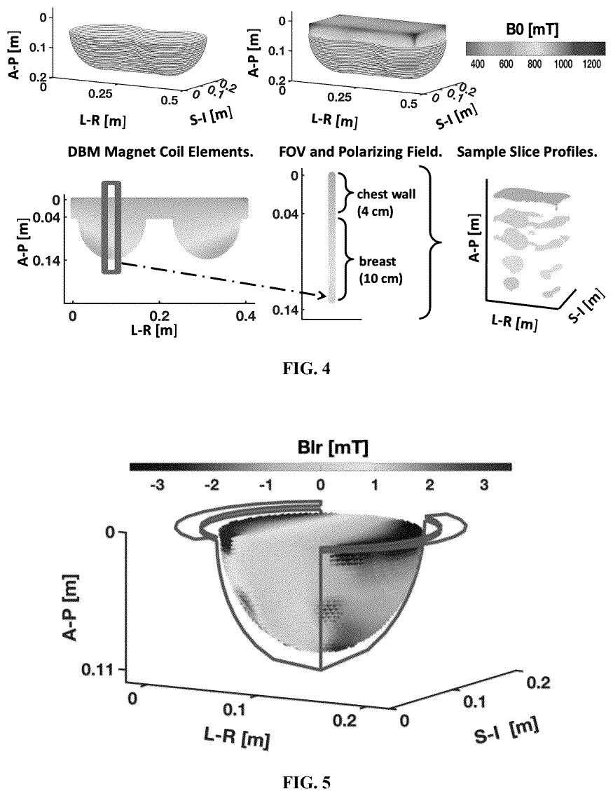

The DBM magnet (FIG. 4) is based chiefly on a solenoid coil design which is typically wound in a helical or cylindrical shape, which creates a strong magnetic field in the A/P direction (recall that NuBo has B0 in the L/R direction). Modifying a straight cylindrical solenoid coil into a shape resembling a bra-cup and varying the density of the coil windings with depth we can produce a fairly uniform field (not a requirement, but it makes things easier) and extend this volume above the magnet to include the chest wall. While RF coils are designed to be compact and close to the breast, magnets must have a larger diameter to accommodate the gradient and RF coils within. To generate a strong magnetic field, these magnets are constructed by stacking several layers of thick wire to carry high current. Consequently, it becomes difficult to meet the distance requirement between the two breasts with individual solenoid magnets for each breast. To address this, we designed a modified solenoid coil that encompasses both breasts, as depicted in FIG. 4. A potential advantage of the DBM over the NuBo magnet is shorter wire length. Shorter wire length results in lower resistance in the electromagnet, allowing for reduced power consumption and heat generation and excellent ramp times. This yields a higher B0 field per volume and weight, which is more efficient and lowers construction costs. Simulation results indicate that with the same power amplifier as the NuBo system (Performance Controls Inc) a DBM magnet design with 175 windings (25 turns×7 layers) we can meet these endpoints. We can use hollow wire for windings which allows for highly efficient cooling. In the NuBo system we have only a few degrees increase in temperature running at a higher duty cycle than we would for imaging.

The strength and direction of the magnetic field produced by this magnet are calculated using the Biot-Savart law. As shown in FIG. 4, the region of interest (ROI) for the DBM system is like that of the NuBo system, with dimensions of 40 cm×20 cm×4 cm for the chest-wall and two cup-shaped breasts with 16 cm in diameter and 10 cm in height. The DBM generates a strong polarization field throughout the imaging volume including the chest wall area, with field strengths from 0.4 T to 1 T in the breast and chest wall regions.

Non-Limiting Acceptance Criteria for this Breast Magnet Include:
Ramp time of 25 ms from 0 to max 1 T.
Maximum temperature allowed 37° C.
Maximum heating rate allowed 0.5° C./s below 30° C. and 0.2° C./s above 30° C.

The maximum rise time and stability can be measured following a ramp up or down, and in safety tests to ensure that these maximums do not induce any peripheral nerve stimulations (PNS) in subjects. Just as in the NuBo magnet, the non-uniformity in the magnetic field can be harnessed as a slice-select gradient in the A/P direction. Because of the difference in the B0 magnetic field direction between NuBo and the DBM, the gradient and RF systems cannot be shared across these designs.

### A.3 Local Gradient Coils

By designing local gradient coils that surround a breast of interest, we can achieve high spatial resolution with low-cost gradient coils and amplifiers. Slice selection with our field-cycling magnet is performed by adjusting B0, and then in-plane spatial encoding is performed using local gradients and receivers. In the specific instance of breast anatomy, each breast has its own set of spatial encoding gradients, and FOV, but the acquisitions are performed in parallel. This has advantages in terms of resolution and acquisition time and represents another form of parallel imaging. The designs of our unique gradient systems are innovative and tailored specifically to the B0 field produced by the field-cycling magnet.

### Spatial Encoding Gradients for DBM

Since the direction of the magnetic field of the NuBo magnet and DBM differs, the gradient coils used with the DBM must be designed differently from those of the NuBo. Since the DBM system is built using a solenoid coil with B0 aligned with the axes of the solenoid gradient designs are more like those of a conventional commercial high-field MRI, with the caveat that rather than the imaging volume being in the middle of the solenoid, it is at the end and extends beyond the solenoid. AP gradient coils can be designed similarly to Maxwell coils. However, as the DBM itself contains the AP gradient field, only the LR and SI gradients are required. These LR and SI gradient coils can be designed similarly to Golay coils. FIG. 5 illustrates the gradient coil design accounting for imaging at the end and even beyond the edge of the magnet. The loops on the anterior side are designed to match the shape of a bra cup, while the loops on the posterior side are calculated using the Biot-Savart law to create the desired field gradient. Simulations indicate that this design produces a relatively linear gradient field-noting that we do not need the gradients to be linear as we know how to image with nonlinear gradients⁷⁷⁻⁸¹. While only the LR gradient coil is depicted in FIG. 5, the AP gradient coil is implemented with a similar coil rotated 90 degrees about the vertical axis.

Preferably, in-plane spatial encoding gradients that generate a gradient of at least 40 mT/m with 25 amps of current are desirable.

### A.4 Local Transmit and Receive RF Coils

The disclosed approach to RF is innovative in that custom coils are designed complementary to the B0 main magnet. Initially we can work with pairs (one for each breast) of transmit and receive (Tx/Rx) volume coils that are designed to encompass each breast and provide good sensitivity to both the breast and the chest wall. Receive-only array coils for accelerated parallel imaging can also be implemented in the breast MRI systems. Parallel receive coils could provide advantages in SNR and accelerated acquisitions²²⁻²⁵ (particularly relevant for dynamic contrast enhanced (DCE) imaging) further enhancing our system. Suitable arrays, such as a hammock-array (for the NuBo system) and a circular array (for the DBM system) described below, can be implemented for this application-again with a separate RF setup for each breast. These RF coils reflect innovations necessary to image in these non-standard magnetic fields.

### Transmit and Receive RF Coils for DBM

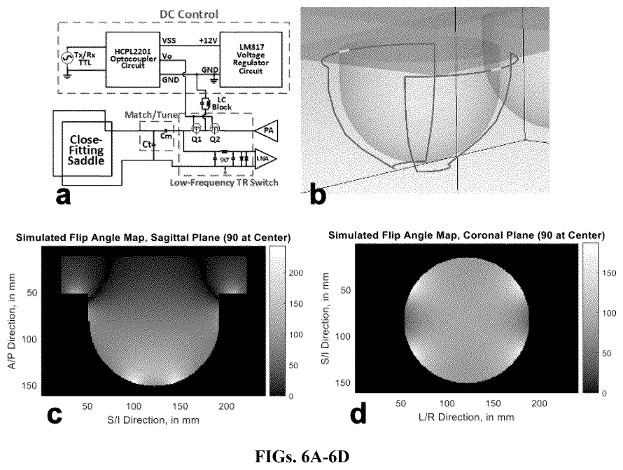

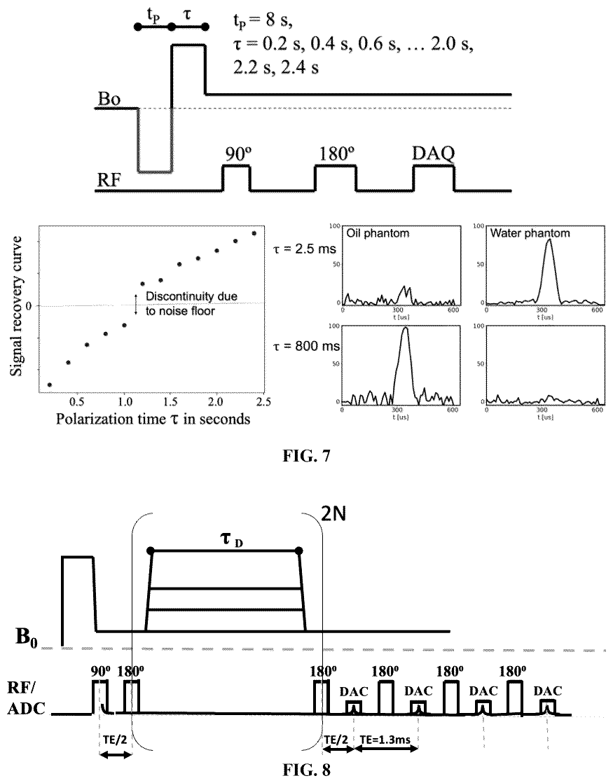

As with NuBo, initial studies on the DBM system, can use a single channel Tx/Rx coil. We have designed a customized-shape volumetric Tx/Rx saddle coil to generate a B1 field transverse to the B0 (which is in the A/P direction), as shown in FIGS. 6A-6D. For a design that is compact and maximizes the filling factor, the saddle coil is designed to follow breast geometry and is tapered at the bottom. The theoretical maximum uniformity⁸² is achieved by making each half of the saddle 120° for any coronal plane.

### A.5 Console

While our initial systems can use commercially available Tec-Mag Redstone consoles, we can develop a low-cost console similar in nature to the OCRA open-MRI consoles currently available^{26,105-108}. To purchase a console from a major manufacturer can cost 100's of thousands of dollars. Yet it has been shown that programmable circuit boards can be used to develop controller hardware for MRI systems for less than $1000. Following the developments from the open-source console group (OCRA)²⁶ a field programmable logic (using Verilog²⁷ programming) can be used as the basis for a console for our system. This can be interfaced with iOS iPad using the Apple Swift code for open-source development, representing another innovation.

The console controls the pulse sequences, which control the polarizing field, the spatial encoding gradients, the RF, and the data acquisition boards. It controls the timing of the transmit radio frequency (RF) pulses through the hardware and receives and processes signal from the hardware to generate images. Commercially available MR consoles, like TecMag's Redstone, are high performing but also inflexible, expensive, and technically opaque. As a result, many ‘homemade’ consoles¹⁻⁶ have been designed which offer a more configurable, low-cost design to accommodate research and development applications. For this purpose, field programmable gate arrays (FPGA) have become popular in console design because they are reprogrammable and can process data in parallel making them flexible and well suited for the requirements of MRI. While FPGA based consoles have been designed previously to improve console performance^{1,2}, many designs revolve around a central data bus and thus lack scalability. However, many research and commercial MR applications demand that consoles can handle multi-channel parallel transmit/receive RF and data acquisition systems. Existing implementations of multichannel consoles split the RF signal generated on a single FPGA equally into vector modulators for each channel^{3,4}. While this design allows for a scalable design, it lacks flexibility because all channels must operate at the same frequency and use the same pulse sequence. The Medusa console¹⁰⁷ offers a modular design with individual channel control of frequency and timing but uses analog RF hardware which is not ideal. There is another scalable console which solves this by using entirely digital hardware¹⁰⁸, but it only includes the receive path and thus has no pulse sequence design. We can combine these innovations to develop a completely modular FPGA based console, allowing a scalable number of independently controlled transmit and receive channels to be configurable to new developments in MR hardware and pulse sequences.

### Complete the HDL Programing for Controller, Magnet Control, and Tx/Rx Boards

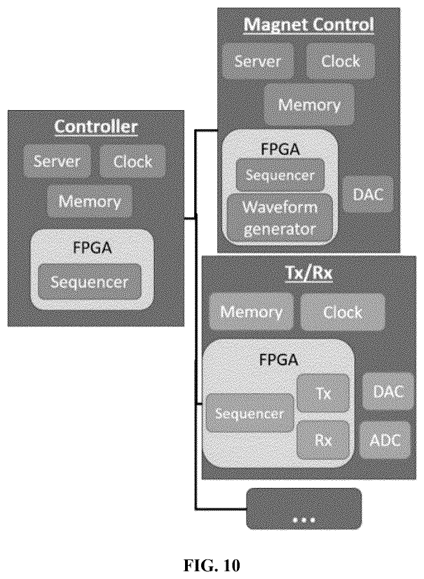

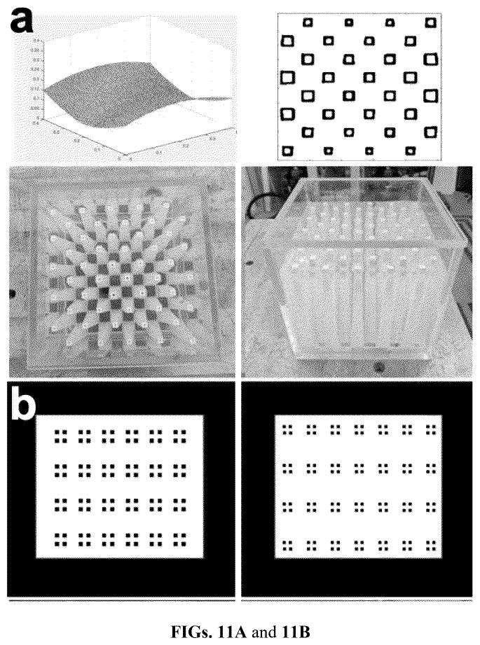

A block diagram level schematic of the logic distribution across the components of the console has been developed as shown in (FIG. 10). This modular design results in the most flexible console, as well as the least expensive console because the FPGA work is shared across several boards to avoid one large, expensive FPGA. To expedite this step, we can maximize the use of open-source hardware description language (HDL) code as possible from. The transmit (Tx), receive (Rx), and waveform generator logic can be identical to these previous open-source projects as it already has been proven to be effective. The data path from the controller board to the peripheral boards (magnet control and each Tx/Rx) is new. A controller sequencer HDL program can be created which can send the individual channel pulse sequence to each applicable board serially. Updates to the sequencer logic for the Tx/Rx boards and magnet control boards can add an additional control step so sequences are not executed until an additional command is received from the controller board. Thus, each peripheral board is executing independently, increasing the modularity of the design. On the receive path data can flow to each Tx/Rx board for initial storage data until it receives a command from the control board requesting the data to limit parallel processing power needed from the controller board. Once completed, all HDL modules can be made available via GitHub. The resulting design can be easily scalable to as many channels as the number of outputs on the controller board allow. The original design can accommodate up to 32 transmit/receive channels for the RF.

### Build a Prototype of the Console

Starting from the block diagram and system level requirements for the console, all hardware requirements can be derived. This step can include configuration of the size of the FPGAs to minimize cost and size of the system as well as decisions about the specific FPGA chips and other hardware that can be used. This step can finalize the number of channels per board. An online schematic tool-Digi-key's “Scheme-it” can be used to create the circuit schematic. We can then build a simplified prototype of the console with evaluation board versions for each of the components. Initially, we require only a two Tx/Rx boards as opposed to the full 32 channel Tx/Rx design. This can be simple to build and easy to troubleshoot. This can be sufficient to test the modular logic and timing of the design. The hardware and logic design can be evaluated using this prototype.

### Pulse Sequences and User Interface

Our pulse sequences can be ported from a console (such as the TecMag console) to an alternative low-cost console. The software for the user interface can also be developed and we envision this to be developed on a device (e.g., a hand-held device such as a tablet (e.g., iPad), mobile phone (e.g., a smart phone) or other hand-held devices). The user interface can have only very limited control-allowing the user to start and stop a study or scan, and to select which pulse sequence to run from a fixed order menu. In addition to prompting for each scan it can include prompts to begin contrast injection. Preferably, none of the pulse sequence parameters will be available for modification by the users unlike on conventional magnets. This ensures that the proper protocol is always applied, and data consistency is maintained to facilitate machine learning and/or deep learning AI assisted radiology.

A.6 System Developments Herein that are Applicable to Many B0 Geometries

### Pulse Sequences

Field cycling introduces new contrast mechanisms and because a polarization period must be added to each sequence many new aspects must be introduced to utilize this technology. We describe below the development of T1-weighted, T2-weighted, and dynamic contrast enhanced imaging, sequences to integrate with our field cycling system. We also include fat saturation and new developments in diffusion imaging as a component of breast MRI exams. Two major innovations on the pulse sequences side include the use of B0 field-cycling for generating strong diffusion weighting with very short Time to Echo (TE), and the reversal of the polarizing field during the initial polarization period for nulling the signal from fat (or other tissue present in the sample/volume of interest).

Conventional pulse sequences and contrast mechanisms can be accessible with the disclosed breast MRI systems. The described protocols closely match those used at standard clinical settings with 1.5 T and 3 T. This includes T2-weighted turbo-spin echo, diffusion weighted imaging (DWI), T1-weighted TSE (pre, dynamic T1, and post contrast administration to look for late enhancement)⁸. Our target resolution can be a slice thickness of 2.5 mm and in-plane resolution 1 mm2⁸³. While this can be developed for in vivo application, our target contrast can have a max dose of gadobenate dimeglumine 0.1 mmol/kg of body weight^{84,85} with power-injector 2 mL/sec flushed with 20 mL bolus saline. We can use fat suppression and explore DWI of breast, while maintaining short TE. This is possible because our magnet can produce a very large gradient (b-values even over 1.4×10−3 mm2/sec8) in a short encoding period minimizing TE and maximizing signal.

### T1-Weighted and T2-Weighted Imaging

Both T1- and T2-weighted sequences can be turbo-spin echo sequences for minimizing scan time. T1-weighted sequences are very useful in lesion detection^{10,86} and also serve as the baseline image prior to contrast agent administration. T2-weighted sequences capture breast edema, hemorrhage, mucus, and cystic fluid^{10,86} The T2-weighted images can reduce the number of false-positives, and also allow for well-circumscribed cancers to be differentiated from benign breast masses. We can perform T2-weighted imaging both with fat suppression (fluid intensity is intensified) and without fat suppression (quality of tissue architecture and lesions morphology is enhanced).

We can implement a T1-weighted turbo-spin echo pulse sequence for dynamic contrast enhancement studies with a maximum acquisition time of 100s and repetitions for up to 6 minutes post-contrast injection. Our target resolution can be a slice thickness of 2.5 mm and in-plane resolution of 1 mm².

### Contrast-Enhanced Sequence Development

T1 contrast agents are sensitive for the detection of breast cancer as the intravenous contrast agents are distributed through the extracellular space and accumulate in areas of high blood flow^{8,87}. To maximize the T1-imaging capabilities, we can use gadobenate dimeglumine which has an in vivo T1 relaxivity that is twice the T1 of the widely used contrast agent gadopentetate dimeglumine⁸⁸. We can use a maximum dosage of gadobenate dimeglumine 0.1 mmol/kg of body weight^{46,47} with power-injector 2 mL/sec flushed with 20 mL bolus saline. Breast images can initially be acquired prior to IV injection and then 100, 200, and 400 seconds post-injection, though optimal timing can be determined experimentally by acquiring breast images with high temporal resolution in 4 subjects with known pathology. The endpoint of this task is to implement a serial multi-phase acquisition with short acquisition time in our T1w acquisition such that breast images can be acquired prior to IV injection and then 120-, 240-, and 360-seconds post-injection.

### Field Inversion for Nulling Fat or Water

Field cycling also can be used to exploit T1 contrast as the polarization accumulates at the rate of T1. T1 contrast increases at low field, increasing the selectivity of this approach⁸⁹. FIG. 4 shows how reversing the polarization field direction with a variable duration acts much the same way as conventional inversion recovery imaging allowing the contrast between two different T1 species to vary as a function of negative- or positive-polarization time. Preliminary data indicates that this provides, a T1 dependent polarization curve for each spin species, rather than the conventional T1 relaxation curve.

### Diffusion Weighting

Diffusion-weighted imaging, unlike other conventional contrast imaging, has high sensitivity to the detection of changes in the cellular environment without the need for intravenous contrast material injection. While the spatial encoding gradients and echo spacings during readout at 24 mT provide little diffusion weighting (b<2 s/mm2), strong gradients are available from the main B0 magnet. The pulse sequence diagram in FIG. 8 shows an innovative approach we have developed to use this capability to provide a diffusion weighting preparation module. Preparation echoes can encode diffusion with gradient amplitudes >600 mT/m. This encodes b˜6000 s/mm2 with one pair of 7 ms pulses, providing very strong diffusion weighting while maintaining short TE for maximum signal. Note that higher b-values have been shown to improve lesion conspicuity^{90,91}.

Diffusion weighting can also be increased by applying serial shorter diffusion encodings (i.e., N>1 in FIG. 8). Notably, the field (and gradient) during diffusion encoding can be either higher or lower than the 24 mT readout gradient, so b-values as low as 5 s/mm2 can be encoded over the fixed echo time. Encoding multiple diffusion weights at constant echo time aids quantitative data models. With appropriate timing, a refocusing pulse train can directly begin the readout and spatial encoding. In addition, multiple modules can be inserted within the echo train, potentially yielding data at multiple b-values in each repetition time (TR).

### Image Reconstruction Software

Breast reconstruction algorithms can be customized to accommodate the spatial encoding and data acquisition schemes that arise from different gradients and RF. Note that we do not use standard Fourier based image reconstruction because that cannot accommodate the nonlinear spatial encoding fields that are used for spatial localization. We have extensive experience with spatial encoding using nonlinear gradients and some of the efficiencies that this brings to encoding^{77-81,92-98}.

### Eddy Current Corrections

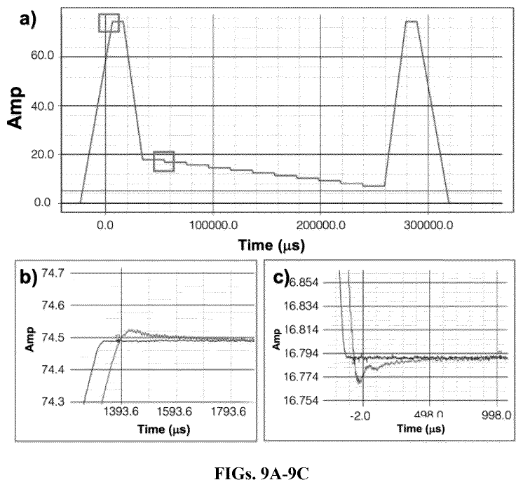

The polarizing magnet used in both NuBo and DBM is essentially a very high-powered gradient, and as such, sophisticated corrections like those used in traditional gradients are possible and appropriate. One potential concern is eddy currents, i.e., fields associated with currents induced during switching. Our measurements from a simple trapezoidal input waveform shows short stabilization times when ramping down from polarization to imaging field on the NuBo system (see FIGS. 9A-9C). However, measuring gradient impulse response functions (GIRF) in strong nonlinear gradients can be done, and standard methods work well. We have verified that GIRF characterization of nonlinear gradients can provide successfully pre-emphasized waveforms^{99,100}. For eddy currents associated with diffusion imaging, modern eddy correction post-processing techniques used for DWI image processing already model the eddy currents with a nonlinear basis, and these techniques are also effective¹⁰¹.

### Concomitant Gradient Corrections

Concomitant terms from spatial encoding gradients are another important consideration. During the low readout field, even weak spatial encoding fields can have non-negligible amplitude compared to the static magnetic field. Therefore, components perpendicular to the main magnetic field contribute to the Larmor frequency and must be factored into the phase evolution¹⁰². Fortunately, our calibration approaches can provide spatially (and temporally) resolved measures of B_{LR}, B_{SI}, and B_{AP} for both the main field and the spatial encoding fields, so corrections associated with concomitant fields are straight forward to calculate and these can be incorporated into the encoding matrix for reconstruction. Our reconstruction algorithms can be extended to include the effects of concomitant fields. The effect of concomitant fields can be estimated from initial measurements of the magnetic field in polarizing and gradient hardware. These can be incorporated into encoding matrix to properly model phase evolution during spatial encoding. Initial reconstruction algorithms can use GPU-accelerated algebraic reconstruction to provide maximum flexibility so that all hardware characterization can be accurately incorporated into the encoding matrix.

It is contemplated that the reconstruction time and implementing denoising and regularization can be improved via machine learning. Since our data can be collected with identical imaging protocols, we can rapidly accumulate a large, annotated set of breast images that can enhance the performance of deep learning strategies for artifact reduction and image quality improvements. We can also develop software to subtract the pre- and post-contrast images to generate maximum intensity projections for rapid lesion detection^{103,104}.

### Compare NuBo and DBM Prototypes and Choose the Best System

Data from human and phantom studies can be analyzed for polarizing field, field uniformity and coverage (including chest wall and axillary regions) gradient strength, resolution, SNR, contrast, and stability. The best preforming scanner on these metrics (with maximum weighting given to resolution and SNR (since many factors such a field uniformity, polarization strength and gradient strength combine in these summary measures of SNR and resolution)) can then be further developed for deployment in a clinical environment and subsequent clinical efficacy studies.

We have constructed a pyramid phantom (FIG. 11A) that provides a useful shape for quantifying image sharpness across the field-of-view as well as for measuring the slice profile. Our slices are non-planar and thus the size of the squares in the image allows us to calibrate the slice profile (as measured vertically from the flat surface of the magnet) as a function of position in the FOV. Using this phantom, we can calibrate slice profiles and measure the sharpness across the FOV. Quantifying sharpness of the gradient along each of the 4 edges of each pyramid, provides a 2D view of sharpness throughout the FOV. Signal can be measured between every pair of pyramids and then fit to a smooth surface to yield SNR across the FOV. We can also use a series of three small contrast detail phantoms (two are shown in FIG. 11B) constructed of a plexiglass box with vertical square rods in sets of 4 with 3 mm skip 3 mm (in one phantom), 2 mm skip 2 mm (in another), and 1 mm skip 1 mm rod placements (in a 3rd phantom). These phantoms can provide a measure of spatial resolution across the FOV. Each phantom study can be repeated three times to provide an average value for each metric. The endpoint is to resolve the 2 mm rods with at least 50% contrast. Improvements of pulse sequence parameters, views, timing, number of encodings per polarization period can be performed in phantoms. These improvement studies serve to maximize image quality with depth, contrast, and SNR.

A set of contrast phantoms can also be developed to assess adequate contrast is achieved for each pulse sequence. All phantoms can be designed to cover the FOV of a breast, both breasts, chest wall, or a combination thereof, such as both breasts and chest wall. The phantom used to assess T1 contrast can contain vials doped with gadobenate dimeglumine to mimic relaxivities observed in vivo. Similarly, a phantom for T2 contrast can contain multiple concentrations of MnCl2, a phantom for diffusion contrast can contain multiple concentrations of PVP, and a phantom for fat/water suppression can contain vials of oil and water.

Each scan optimized in phantoms can also be tested on healthy volunteers. These images can be examined for perceived artifacts and appropriate contrast both in breast and chest wall. These studies can also be used to inform appropriate protocols for human scanning, including ergonomics.

Spatial resolution as a function of encoding strategy is our primary outcome measure and thus the best acquisition strategy can be that with the best average resolution (as measured using the contrast detail phantom) over the 16×16 cm2 FOV. Strategies that also yield excellent SNR, and image sharpness can be preferred after resolution is maximized. Contrast is assumed to be highly similar in NuB0 and DBM scanners, but discrepancies can be quantified and taken into consideration. In addition, scans from healthy human testing acquired during method development can be reviewed by radiologists on our team and rated on a Likert scale for overall image quality to ensure that the system yielding highest resolution is also the one found to have highest clinical quality.

### A.7 MRI Siting, Installation, and Operations

Imaging sites (ranging from breast clinics to franchises such as CVS or Quest Diagnostics) would provide retail space for the exams, etc. Secondly, all the data can be routed to a central reading resource where radiologists perform the read. This has two primary advantages: i) the community sites do not require radiologists; ii) the standardized data from many sites rapidly creates a large training set for machine learning and/or deep learning AI radiology applications focused on AI-assisted radiology³, such that ultimately many cases can be read quickly by few radiologists further decreasing costs.

Also contemplated are additional developments that could incorporated in extensions of these breast MRI systems, which facilitate different applications. For example, both the magnet and the RF can be swapped out for imaging different anatomic structures, but with the same amplifiers, chiller, and other components. Where these swappable front ends were in different rooms, the breast MRI systems can be used in a multiplexed fashion across rooms greatly saving costs on amplifiers and other components.

### III. Methods of Using

Also disclosed are methods of breast-specific imaging using the breast MRI systems described herein, the methods involving field cycling between the first non-uniform B₀ magnetic field and the second non-uniform B₀ magnetic field. The first non-uniform B₀ magnetic field is applied to the sample/volume of interest prior to applying the second non-uniform B₀ magnetic field to the sample/volume of interest, and field cycling between the first non-uniform B₀ magnetic field and the second non-uniform B₀ magnetic field is achieved by controlling/altering a current supplied to the magnet. RF coils can be used to generate spatial gradients, to select a non-planar isofield slice, and receive mode (e.g., parallel receive mode) to detect magnetic resonance signals. Each of these tasks can be performed using either the same or different subsets of RF coils. DC nonlinear gradients can be used for spatial encoding, either with or without the addition of RF encoding and/or receive mode (e.g., parallel receive mode). Breast MRI images are reconstructed from (potentially parallel) received magnetic resonance signals from RF coils using algebraic reconstruction, preferably without the use of standard Fourier-based image reconstruction. Preferably, the breast MRI systems are designed for breast-specific imaging.

The ensuing non-limiting section provides an in-depth discussion of applications to MRI for breast screening. It is noted that given these discussions and technical details, the methods can be readily adapted to other anatomic areas including, but not limited to, those described herein.

### A. Breast-MRI

Breast cancer continues to be a significant global health concern, affecting millions of women worldwide. Early detection plays a crucial role in improving outcomes and reducing mortality rates^{4,5}. Mammography has long been the gold standard for breast cancer screening, despite poor sensitivity⁶. MRI has emerged as a promising technology for breast screening mammography^{7,8}. The sensitivity of MRI in women with various risk profiles ranges from 81% to 100% which is approximately twice as high as the sensitivity of mammography in these groups. While the positive predictive values for biopsy are in the same range as mammography, the specificity of imaging increases in follow-up rounds to 97%⁹. This performance implies that all other examinations should be regarded as supplemental to breast MRI⁹. However, a primary limitation to using MRI in general as a regular screening modality is the lack of access and high expense. It is important, therefore, to develop an accessible breast-specific MRI system designed to move MRI out of the large radiology centers and distribute them widely throughout the community such that breast-specific MRI can become part of a regular annual checkup for individuals, particularly women in the context of breast-specific MRI. Such a breast MRI system offers several technical advantages for diagnostics, e.g., breast cancer screening:
(1) Enhanced Sensitivity: MRI has shown superior sensitivity in detecting breast tumors, especially in women with dense breasts⁹⁻¹¹. Fibroglandular breast tissue can mask abnormalities on mammograms, making it challenging to identify potential cancers¹⁰⁻¹³.
(2) Improved Accuracy: The high resolution and superior soft-tissue contrast capabilities of MRI provide a comprehensive view of breast tissue. MRI can help differentiate between benign and malignant lesions, reducing false-positive results⁹. By minimizing unnecessary biopsies and interventions, MRI can alleviate patient anxiety and prevent potential complications. Open MRI systems and the lack of needed breast compression as in the proposed device can also reduce patient anxiety associated with such screening tests and greatly improve patient comfort. AI assisted³ breast MRI in the context of an annual exam aimed to detect change could potentially provide even more benefits.
(3) Detection of Additional Cancers: MRI can detect additional malignancies that may be missed by mammography^{14,15}. Multiple studies have demonstrated that MRI can uncover occult cancers in the opposite breast or in the same breast but different quadrants, enabling comprehensive evaluation and treatment planning. This capability is especially valuable for women at higher risk of developing breast cancer.
(4) Early Detection in High-Risk Populations: MRI is particularly beneficial for individuals with a high risk of breast cancer due to genetic mutations (e.g., BRCA1 and BRCA2)¹⁶. These individuals often develop aggressive tumors at a younger age. The increased sensitivity of MRI can facilitate earlier detection, leading to more effective treatment options and improved outcomes. The lack of radiation associated with an MRI exam also makes including this as part of an annual checkup attractive.
(5) Assessment of Treatment Response: MRI can play a crucial role in monitoring treatment response and evaluating tumor size reduction following neoadjuvant therapy. By accurately assessing tumor response, clinicians can adjust treatment plans and optimize patient care. This capability of MRI helps in tailoring personalized treatment approaches, resulting in improved patient outcomes.

While MRI offers numerous advantages, certain challenges and limitations still remain that the disclosed breast MRI systems seek to address: First and foremost, MRI in its conventional form is simply too expensive, limiting its widespread use and accessibility particularly in resource-limited settings. By making an inexpensive dedicated breast MRI system enabled through several innovations, we can make an affordable MRI that will greatly increase accessibility. Further, the requirement for specialized expertise and dedicated infrastructure adds to the financial burden. Preferably, the disclosed breast MRI systems are designed to be operated by selecting a few buttons on a device (e.g., a hand-held device such as a tablet (e.g., iPad), mobile phone (e.g., a smart phone) or other hand-held devices) and combining this innovative technology with remote medicine such that these breast MRI systems can be made available in underserved communities, as well as in diagnostic clinics in small towns across the country and/or the world.

A second challenge present with any screening system, is that of false-positive results. Although MRI improves accuracy compared to mammography^{9,15}, it can generate false-positive results, leading to unnecessary follow-up procedures and potential patient distress. Striking a balance between sensitivity and specificity remains a challenge, and ongoing research aims to refine MRI protocols to reduce false-positive rates. In addition to improved protocols new AI assisted radiology^{3,17} is helping to reduce the number of false positives and as more women are screened more often, these tools will only improve¹⁸. Furthermore, in an annual monitoring environment (say women getting a breast-MRI with each annual checkup), the AI problem becomes a change detection problem, which is a much easier problem to solve than creating a diagnosis from a single scan.

A final challenge that MRI (e.g. breast-MRI) faces is time and resource intensive exams. MRI examinations typically take longer than mammography, and the interpretation of the images requires expertise in breast imaging. These factors limit the throughput and scalability of MRI as a population-level screening tool. However, the disclosed breast MRI systems are designed for rapid setup, abbreviated protocols with short scan times, and ultimately AI assisted workflows to address these limitations, making annual screening MRI more efficient and practical. Our approach to providing low-cost breast-specific MRI systems and new AI approaches will aid in interpretation^{19,20}. An aim is to develop a screening device with a standardized protocol across all sites (to enhance future developments in AI assisted radiology) a centralized reading service (such that a radiologist is not needed on site), greatly easing widespread dissemination of this technology. Also note that dedicated breast MRI systems will make it easier to take advantage of the 10-minute breast protocols that have been shown to be highly effective, replacing the conventional MRI workflows, increasing throughput, and thereby further increasing access.

A centralized reading service with standardized protocols can also rapidly generate very large data to facilitate the training AI algorithms^{19,21}. It is believed that in the coming years radiology will see a large push to standardize protocols to take advantage of new developments in machine learning and/or deep learning assisted radiology.

Breast MRI is a highly sensitive modality which is already recommended for many women²⁹ and likely could benefit many more. An abbreviated MRI exam (<10 minutes) was shown to drastically outperform not only mammography but also state of the art breast tomosynthesis in high-risk women³⁰. Even in women of normal risk, a supplemental MRI screening of 2120 women found more than 60 cancers that were missed by ultrasound or mammography³⁰. Yet access remains a major obstacle. Women who receive breast MRI are generally better educated and wealthier^{31,32}. In addition, travel times to access MRI are longer than for x-ray mammography, particularly among rural residents and minority women³³. A key factor limiting access to breast MRI is concern over the cost-effectiveness³⁴ of this highly sensitive but expensive imaging method.

For 50 years, the steady trend in MRI technology has been towards higher field and more sophisticated (read: more costly) equipment. However, thanks to the many technological advances realized in that time (e.g. major developments in hardware design, spatial encoding, image reconstruction), it has become feasible to design extraordinarily low-cost and accessible MRI hardware with high clinical utility. These systems have potential to disrupt the field and make MRI available in a wide range of settings, including neighborhood clinics, rural communities, and underdeveloped countries.

To date, most of the designs costing less than $500k have several common features. They use arrays of permanent magnets arranged around a cylindrical former to generate a relatively homogeneous B0 field in the center. Because of the limitations of this design, it is difficult to achieve more than ˜65 mT. In addition, the homogeneous imaging volume is limited to the center of that cylinder. Most of these systems also use conventional image encoding strategies, namely linear gradients for slice, frequency, and phase encoding³⁵⁻³⁸. Several low field MRI designs have been demonstrated, both in research and commercially, for brain imaging. A single sided low-resolution MRI, suitable for image fusion in biopsy guidance, has also been developed for prostate. However, there are currently no instruments being developed for accessible breast MRI. As high field MRI scans are approximately 10× more expensive than mammograms, equipment that facilitates affordable breast MRI is key to making MRI a routine modality for either screening or supplemental imaging.

Breast MRI is undisputably the most sensitive method for breast cancer detection³⁹, exhibiting sensitivity approximately twice⁹ as high as standard mammography for many groups. Comparisons of MRI to digital breast tomosynthesis have yielded similar results³⁰. The specificity of MRI is often considered a limitation (83-98.4%), but this ignores the importance of subsequent scans which can be used to calibrate a given MRI. In studies reporting on both prevalence and incidence screening, specificity climbs to 90-97%, suggesting that serial MRIs can achieve both high sensitivity and specificity.⁹ Increasing roles for AI in image analysis are likely to further improve specificity by factoring in previous MRI scans and reduce false positives²⁰.

Ultrasound has been studied as a supplement to improve mammography, particularly in women with dense breasts⁴⁰. However, the added value of ultrasound disappears when compared to MRI screening⁹. For example, one study found that supplemental incidence-screening ultrasound identified an additional 3.7 cancers per 1000 screens, whereas the supplemental yield of MRI was 14.7 cancers per 1000. Ultrasound did not identify any cancers not seen by MRI⁹.

In women with elevated risk, breast MRI is an established and well-justified detection tool, already recommended by most national and international guidelines. Yet even among women currently recommended to MRI, studies indicate that only a small fraction are finding access to those exams⁴¹. One analysis of community utilization found that only 29% of women for whom MRI is recommended actually receive this exam⁴¹. In contrast, approximately ⅔ of women over 40 have had a mammogram in the previous 2 years⁴². This highlights the lack of access to breast MRI, even among women for whom it is unequivocally beneficial.

In practice, one of the biggest hurdles cited for increased utilization of breast MRI is concerns over its cost-effectiveness^{34,43,44}. Previous work has attempted to address this by introducing abbreviated breast MR protocols, and studies as short as 10 minutes have been shown effective. These methods show equivalent sensitivity and comparable specificity to full breast MRI exams, making them attractive strategies for increasing utilization of breast MRI. However, the high equipment and facility costs associated with high field whole body scanners remain a barrier to greater access to this method: The problem this project is aimed at solving.

Because the additional vasculature needed for a tumor to grow beyond 2 mm is often leaky, contrast enhanced-MRI (CE-MRI) is uniquely sensitive to aggressive and clinically significant tumor types. However, non-contrast methods such as diffusion weighted imaging (DWI) have found an increasing role⁴⁵⁻⁴⁷. Whereas CE-MRI highlights vascular features of lesions, DWI provides specificity to the hindered diffusion associated with proliferative pathologies. Adding DWI to the breast MRI protocol, especially a high b-value image, dramatically increases specificity^{48,49}. It may also have value as a standalone exam that avoids contrast injections. Results from non-contrast studies (diffusion and T2w imaging) showed equivalent sensitivity and specificity⁵⁰ to CE-MRI, albeit with reduced lesion visibility.

The promise of breast-MRI is undeniable. Its ability to overcome the limitations of x-ray mammography, particularly in women with dense breasts, holds immense potential for early detection, accurate diagnosis, and improved treatment planning. MRI can provide effective breast cancer screening and contribute to reduced mortality rates. Addressing the challenge of cost, and accessibility, will allow the full potential of breast MRI to be harnessed for the benefit of women's health.

### IV. Methods of Making

The disclosed breast MRI systems can be produced and tested in one or more phases described below, with an important endpoint being the development of low-cost MRI systems for wide-spread screening, diagnoses, and/or monitoring of specific anatomic structures, e.g., for breast cancer screening, diagnoses, and/or monitoring. These breast MRI systems would greatly increase access to this important modality.

### Phase 1: Hardware Development Phase

We have an existing low-cost, field cycling magnet that works. Phase 1 focuses on customizing/improving this system for breast MRI. This process chiefly includes developing breast-specific spatial encoding gradients and RF transmit/receive coils to enable the existing system to perform at high-resolution with high-SNR. This is referred to as the nonuniform B0 (NuBo) magnet. The B0 field direction for this system is in the Left/Right direction, relative to a patient laying prone on the magnet, which requires custom gradient and RF designs.

With the knowledge gained from the development of the NuBo system magnets with geometry appropriate for different applications can be developed, such as a dedicated breast magnet (DBM). The primary B0 field direction for this magnet is Anterior/Posterior (vertical with respect to a patient laying on top of the magnet).

A first endpoint is to complete construction of these two systems in Phase 1 and directly compare them in terms of cost-to-build, polarizing field profiles, gradient and RF performance across the field of view, and signal-to-noise ratio.

The next step in Phase 1 involves completion of the development of common elements including pulse sequences, console, electromagnetic interference (EMI) correction system, and reconstruction algorithm (e.g., breast image reconstruction algorithm). Whether we move forward with the NuBo or DBM system, there are common elements that must developed, and these include the pulse sequences, the low-cost console, noise cancelation hardware/software so the devices can work outside of a shielded room (again to ease siting and reduce installation costs), and the reconstruction algorithm designed for acquisitions with nonlinear spatial encoding.

Another step in Phase 1 involves selecting a magnet design with improved performance for breast-specific MRI

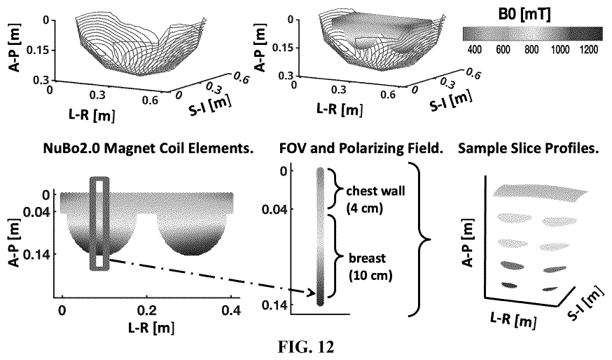

Following completion of the NuBo and DBM prototypes, we can directly compare these systems to determine which one provides better performance for moving forward. The comparison can focus on the polarizing field strength and distribution, imaging performance characteristics, such as SNR, field cycling (stability and speed of ramping up and down), heating, and cost. If the NuBo system is the winner in this comparison, then we can construct a 2^{nd} generation NuBo2.0 magnet, designed and enhanced based on what we have learned so far. NuB02.0, which is designed to provide better polarization, particularly at the chest wall, and provide better ergonomics in terms of access. NuBo2.0 can be constructed following the design outlined below and shown in FIG. 12. We can test this new design against NuBo1.0 and the DBM magnet but are confident it can outperform NuBo1.0 (which would have already outperformed the DBM system). The purpose of this design is to redesign the magnet to be more open and customized to the shape of the anatomic structure (e.g., breast and chest wall), producing a higher field and easier access/comfort for the patient. If the DBM magnet proves to be superior, then we can further develop that design. This is expected to be the only deviation from the prototype unless there are other design modifications that we discover are necessary from the first prototype.

Dependent upon the outcome from the comparison of NuBo and DBM, the next step can be to construct the first clinical prototype and finalize design criteria prior to making additional systems for testing in the clinical environment. Prior to building the 2nd and 3rd magnets for installation in clinics we can do final testing of this first clinical scanner, installed at a patient-only room in the Yale MRRC. We can proceed by imaging 10 healthy subjects in both the prototype and first clinical system. In the unlikely event that image quality is lower than that observed in the prototype, an exact replica of the winning prototype (either DBM or original NuB0 for prone imaging) can be built for installation in clinical sites. Our measurable standards are that the first clinical scanner at a minimum matches performance of the prototype. It is contemplated that this version of the scanner can outperform the original.

### Phase 2: Finalizing Design and Manufacturing Phase

One aspect in Phase 2 would involve defining manufacturing processes for finalized design and building a clinical scanner. Phase 2 focuses on finalizing the overall system design, including the operating software, reconstruction software and all hardware components. This can also include the software for the user interface, safety checks on pulse sequences and the ergonomics of the patient table. Software development can follow an Agile development process based on AAMI TIR 45 which in turn is compatible with the international software standard IEC 6230452.53 Optionally, once the protype design has been finalized this can allow us to engage the FDA in formalizing the construction, quality assurance steps, manufacturing process, and safety. Optionally, the first scanner can be built and validated with well-defined and documented processes in expectation of FDA filing. Careful documentation of all manufacturing, both for the first and subsequent clinical scanners, can be gathered for commencement of the FDA process. This also necessitates formalizing all manufacturing steps and putting in place procedures that ensure quality control in every step of the manufacturing process. Following these steps, we can build two additional systems to be installed in satellite imaging clinics. During this time the technicians on site can be trained to operate the scanner, including all safety aspects, and data archive and transfer steps. The technicians can also be trained to run daily QA procedures on this system-usually at startup in the morning each day.

Another aspect in Phase 2 optionally involves completion of FDA eligible prototypes and clinical implementation. Following validation of the first clinical device, we can build subsequent clinical scanners following the same manufacturing and acceptance procedure. A major step in bringing a new device to clinical use involves engagement of the FDA to begin the process of obtaining FDA approval. The FDA regulates medical devices to assure their safety and effectiveness and this process begins with carefully outlining each component of a complex device, and how it is manufactured in a manner that ensures quality control, both of each component and of the overall device. This milestone completes the initial steps which include the conclusion of device discovery and the construction of a preclinical research prototype. The FDA classifies MRI devices as Class II (moderate risk) medical devices, and thus a 510(k) approval must be sought prior to marketing such a device²⁸. Achieving this milestone means we can begin this process with our breast-specific MRI systems. In addition to collecting data on clinical efficacy of how these systems work, it is first important to install such systems in a clinical environment, such as a mix of academic imaging centers and community imaging clinics.

Another aspect of Phase 2 involves completion of sequences and/or software for monitoring safety and/or for quality assurance. Safety protections can include real-time detection and control of dB/dt, SAR, and temperature for all relevant components. The input from these monitoring components can be interfaced directly to the console to prevent operation outside the safety guidelines. In addition, we can develop a QA protocol to be run on a standard phantom, with automated acquisition and analysis. This can be used to confirm proper operation of all hardware components either on a daily or as-needed basis.

Permanently connected magnetometers can be installed at the edge of each magnet. These, in combination with previously acquired 3D maps, can be used to monitor field stability across time and to prevent scans that may produce PNS. Notably, the maximum slew rate of our systems (˜0.5 T in 35 ms) is still well below the cardiac stimulation limit, which itself is set conservatively by about an order of magnitude. B1 can be similarly monitored using a sniffer coil placed outside the FOV and, in combination with previous measurements, can be used to monitor SAR in real-time. These, as well as temperature monitors for all electromagnets can be interfaced to console which can monitor this input to prevent scans that violate safety limits. It is contemplated that the scanner can automatically detect and prevent scans that violate prescribed dB/dt, SAR, or temperature limits.

A daily scan protocol can be established to confirm proper system function at the start of each day's operation. These scans can be run on a standard resolution phantom with standard positioning, so that images can be automatically assessed. Analyses can confirm that all hardware is within specifications including polarizing field strength, gradient shape, RF power, and receiver SNR. EMI functionality can be confirmed via a scan that excludes signal excitation. The console can run this series consecutively and analyze the data, so no user interaction is required, beyond starting the procedure. If service is needed, the system can automatically send the results to us for troubleshooting. It is also contemplated that scanner can perform scan series and analyzes data in an automated manner. Aside from positioning the standardized phantom and beginning the procedure, no further interaction should be required.

Data collected for safety monitoring, along with environmental variables, can be accumulated in tandem with the daily QA scans. This data can be assembled and mined to identify patterns between QA image metrics and hardware variables. It is further contemplated that contemporaneous data, both images and monitored variables, from all sites can be available and searchable.

Another aspect of Phase 2 optionally involves further advances in SNR and speed. In parallel with system manufacturing, we can test the utility of multichannel phase arrays to implement parallel imaging which can improve scan time. Once clinical data are being acquired through clinical use, we can also test methods to leverage both imaging data and calibration scans to further improve system performance and image quality.

Designs are presented for both the NuBo and DBM systems. However, coils (RF and gradient coils) are different because these two systems have the polarization field B0 pointing in different directions therefore necessitating different designs.

### Develop Parallel Receive Array Coils for Accelerated Imaging

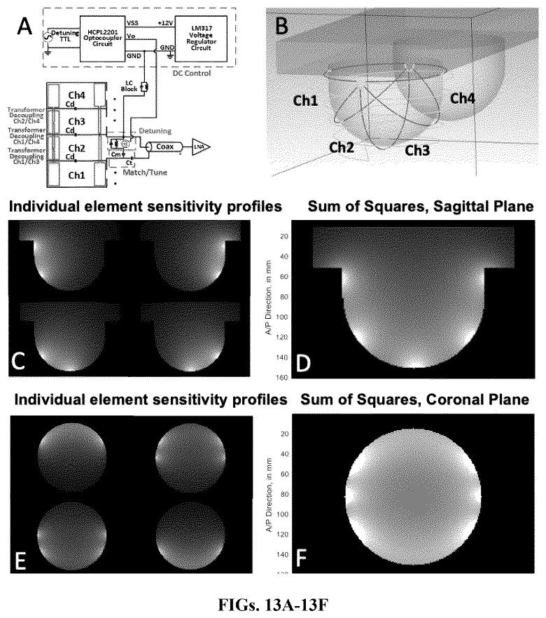

NuBo parallel Rx: Parallel receive coils can exhibit improved SNR along the periphery of the FOV and allow accelerated acquisitions through reduced sampling. We have designed a multichannel Rx array for each breast, with the geometry of a “Hammock” ladder network configuration¹⁰⁹ (FIGS. 13A-13F). This geometry is chosen for several reasons: (1) Since the B0 of the NuBo system is along L/R direction, the majority of B1 field generated by the “Hammock” configuration is perpendicular to the B0 and thus B1 waste can be mostly avoided; (2) So far, the decoupling preamplifier for low field application is difficult to source. The ladder network configuration was originally proposed as a method to achieve, not only adjacent yet also the next-adjacent, coil element decoupling with only capacitive network on one end of the array¹⁰⁹. And in our scenario, both ends of the array elements happen to join together, which conveniently provide a place to put the decoupling network. Besides the original decoupling network, other decoupling such as transformer decoupling¹¹⁰ can also be applied on the two tips of the array. Two elements of the proposed configuration in the next adjacent position were initially built to validate the decoupling method without decoupling preamplifier, as shown in FIGS. 3A-3D. And with the help of the prototype 7-turns transformer canceling the mutual inductance between the two coil, the splitting mode caused by coupling can be mitigated and a 16.7 dB of decoupling can be generated. The subtasks of developing the proposed array coil are listed below:

EM simulation and component value calculation. The array structure can be EM modelled using CST (Dassault Systems Deutschland GmbH). The preliminary B field simulation in Coronal and Sagittal planes were shown in FIGS. 2A-2C. The decoupling network, either with the ladder network¹⁰⁹ or transformer decoupling¹¹⁰, can be firstly calculated based on the simulated coil loss/inductance/mutual inductance.

Bench development and decoupling improvements. The proposed array coil can be built on bench, and the decoupling network between each element can be developed and improved upon. Both ladder-network and transformer decoupling can be investigated. The winner of the two can be used on the final version of the array coil.

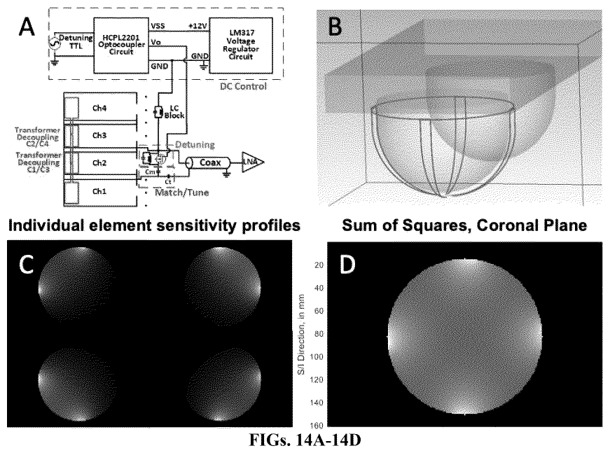

Detuning circuits. TORO configuration requires the Tx coil and the Rx coil to be well decoupled, and this is mostly achieved by detuning one of the two at different Tx or Rx windows. Conventional RF switch design using PIN diodes was found to be too slow for our application, but metal oxide semiconductor field effect transistors (MOSFETs) have been reported to be a good alternative⁷⁵. By combing MOSFET and LC networks, a low-frequency detuning circuit design has been used effectively in a TORO coil configuration⁷³. FIG. 14A shows the detuning circuit that we can use for our coils, comparable to the reported design⁷³.

These designs can give rise to a customized-shape volumetric Tx/Rx solenoid RF coil for breast imaging for the NuBo system and/or a “Hammock” ladder-network multichannel (e.g., 4-Ch) breast Rx-only array coil for parallel imaging for the NuBo system.

DBM Parallel Rx: Finally, a multichannel Rx-only array coil can be built for facilitating parallel imaging in the DBM system (FIGS. 14A-14D). A circular array coil geometry can be implemented here. With the breast shape of the coil, the dome apex naturally provides a convenient point for each coil element to be decoupled, either with capacitor network⁶⁴ or decoupling transformer¹¹⁰.

### The Subtasks of Developing the Proposed Array Coil can be List as Below:

EM Simulation and Component Value Calculation. The array structure can be EM modelled using CST. The preliminary B field simulation in Coronal planes are shown in FIGS. 14B-14D.
Bench development and decoupling improvements. The proposed array coil can be built on bench, and the decoupling network between each element can be developed and improved upon. Both ladder-network and transformer decoupling can be investigated. The winner of the two can be used on the final version of the array coil.
Detuning circuits. Detuning for this array is very similar to that of the hammock-array described above and thus the same circuit will be used.
These designs can give rise to a customized-shape volumetric Tx/Rx Saddle RF coil for breast imaging with the DBM system and/or a circular 4-Ch breast Rx-only array coil for parallel imaging on the DBM system.

### Improve Images Through Accumulated Data

With a fixed protocol on each breast MRI system, we can rapidly acquire a significant amount of data that can allow us to implement reconstructions that better incorporate scanner diagnostic data acquired through QA and system monitoring. We can develop machine learning and/or deep learning approaches to denoising and improving our image reconstructions. It is contemplated that these steps can result in improved SNR over the volume and faster reconstruction times with robustness to artifacts.

### Phase 3: Clinical Testing Phase

Phase 3 involves milestones that can be useful with respect to commencing clinical testing of our breast-specific MRI prototypes.

One aspect of Phase 3 involves clinical data collection. These identical clinical prototypes can be ready for collecting clinical data at the different sites. With systems installed in three sites (two clinical breast imaging centers, and the Yale MRRC) we can begin testing in three different patient groups. The first group includes patients with cancer diagnoses by needle biopsy but prior to any treatment. The second group includes patients who are scheduled to have a breast biopsy based on abnormal imaging (typically mammography and/or ultrasound but also potentially clinical breast MRI). This group can include patients detected at screening as well as those with clinical signs of breast cancer (e.g., a palpable mass). Twenty to thirty percent of this group can have cancer diagnoses confirmed at biopsy, while the others can demonstrate benign lesions. The third group can include healthy volunteers who enroll for screening. This group can provide valuable information on throughput and ease of use of the devices in a healthy screening population. The cancer yield in this group can be less than 1%. For this process we can follow GxP guidelines as specified in ISO 1348551.

Patients with biopsy proven cancers: The first step that any device proposed for cancer screening must pass is to address the question as to whether or not it can detect known cancers. Patients who have had an abnormal imaging study and who have been sent for biopsy which has confirmed cancer, will be recruited and scanned on our device. A team of three radiologists can review the cases and score each study on a 5-point detectability scale (ranging from nothing seen, to some evidence, to very apparent abnormality). If additional lesions are visible, as is often the case in both clinical practice and these types of studies, then the radiologists will rate the detectability of what they consider the most suspicious lesion. While not all patients at Yale have a clinical MRI as part of their cancer workup, for those that do we can compare the performance of the device to that of the commercial 3 T scanner.

Patients who are going for biopsy (not confirmed cancers): This group will also be read by a team of three radiologists and again they will generate two 5-point scales. The first scale will be for scoring on detectability as used in the biopsy-proven cohort and the second will rate the likelihood of malignancy. These patients will subsequently undergo a biopsy and we can confirm the standard measures of true positive, true negative, false positive, and false negative. The rating scales will allow us to generate ROC curves^{111,112} for our device.

Power analysis: For the confirmed cancer group, our primary hypothesis is that the new breast imaging device is either equivalent to or superior to the standard MRI in detecting breast cancer. Based on recent literature⁹, we assume that the average sensitivity of the standard breast cancer MRI is 0.9 for cancer detection. Let δ represent the non-inferiority margin; we can construct a statistical test under the null hypothesis that the cancer detection rate of the new imaging device is smaller than δ compared to the standard one. The alternative hypothesis is that it is smaller or equal to δ. The test statistic will asymptotically follow a normal distribution, determined through a restricted maximum likelihood estimation approach. With δ=0.9, a sample size of 118 patients will provide us with 80% statistical power to conclude the non-inferiority of our new device, at a significance level of 0.05. For the pre-biopsy group, under a 5-point rating scale to classify lesions, we can conduct ROC (receiver operating characteristic) analysis to assess the performance of the new device for cancer detection. We expect 20% of the patients in the pre-biopsy group to have a confirmed cancer. By assuming the AUC (area under the ROC Curve) under the null hypothesis to be 0.7 based on that for the current screening mammography, a group size of 595 subjects will achieve 90% power to detect a difference 0.09 AUC difference using a z-test at a significance level of 0.05. All the power analyses were performed using PASS.

Another aspect of Phase 3 involves assessment of clinical performance. Clinical performance in terms of detecting known cancers (patient group 1) can be assessed by measuring detectability of known lesions. Sensitivity and specificity can be assessed in an enriched population as defined by patients who have had an abnormal clinical exam and are scheduled for biopsy (patient group 2). Data on patient flow and image quality in a screening setting (patient group 3) can also be collected. It is desirable to aim for values of the order of 95% for cancer visibility, but it can be beneficial to be equivalent or better than screening mammography, which is about 75% overall and down to 50-60% in dense breasts.

Another aspect of Phase 3 is having a commercializable prototype with initial FDA engagement and clinical efficacy data that can be translated to a marketable device through transition to industry.

The final product from this work can be a prototype breast MRI system that can have sufficient documentation and data to complete FDA approval and move to market. The system combines many developments in breast MRI disclosed herein in a unique package that allows for the design of a highly efficient breast MRI device. This system can be low-cost, easy to site, and easy to operate. By making such systems available at 1/10th the cost of conventional MRI, we should be able to greatly increase access to MRI breast screening for all women. MRI has been shown to be the best modality for detecting breast cancer and the only reason it is not used routinely and access is very limited is because of the high cost of purchasing and operating a conventional MRI system. The disclosed breast MRI systems can solve this problem and this in turn can have a major impact on women's health and the war on cancer.

The technology we develop for this system is transferable to imaging other organs. In these applications we envision building such devices into the patient beds in intensive care units such that the patients do not need to be disconnected from all of their tubing and moved into an MRI scanner to check on their status. In the intensive care setting each bed could have a local electromagnet and coils built in and these could all be connected to a central setup of power amplifiers, rf amplifier and a chiller. These are the most expensive components of this device and by multiplexing these to many front ends, tremendous cost savings could be had in siting these MRIs.

Moreover, the development of low-cost and safe (no ionizing radiation) MRI could lead to imaging as a routine part of annual exams for all. Specifically for the breast, the low cost and wide access of the breast MRI device will allow for easy annual exams for all adult women. With our approach, all exams can be conducted with identical protocols (technicians do not input any parameters into the scans), and therefore the images can be consistent in size/contrast/etc. The constancy of the breast images and the rapid accumulation of a large database of images over time can allow for the development of AI assisted radiology methods that can detect any changes in the breast images (AI is more efficient at detecting change than making diagnosis). The use of AI in detecting breast changes can also lower the cost of exams by reducing the time radiologists need to spend with each case they read.

By having all exams conducted under the same protocols, and by having all images uploaded to the same database servers, breast evaluations can be done in real time by radiologists stationed in locations outside of the exam centers. This can be especially helpful for areas that lack radiologists, such as underserved or remote communities. In our business model we retain ownership of the MRI devices and hence the data and provide the radiology service. Such an arrangement maintains control of image quality and allows us to quickly build very valuable data for training AI-assisted radiology algorithms.

Future work could also extend these devices to accommodate breast biopsies. The open nature of the breast MRI device allows for easy access to the breast and ensure the correct tissue is biopsied in real-time. This new approach to MRI can also be expanded to other applications—where magnets are designed to fit specific anatomic locations. By installing MRIs directly into the patient beds, patients can be imaged multiple times per day, without needing to move the patients. This could be especially helpful in rapidly progressing health problems where the continuous collection of images yields better health treatments.

A summary providing some more specific, non-limiting aspects of the disclosed breast MRI systems and methods are provided below:

### Phase 1: Hardware and Method Development

### A. Hardware Development

### 1. Gradients for NuBo

1.1. Hardware can be built from 3 mm conductive copper with 35 (Gsi) and 28 (Glr) windings in a single layer for each channel. Following construction, gradients can be tested for resistance, inductance, and flow. They can then be matched and tuned to 25A amplifiers (AE TECHRON) and tested at full current for heat, torquing, and coupling. 3D measurements of the static field for each channel can be acquired (resolving Bx, By and Bz), and we can also perform measurements at multiple locations to assess the field during a short impulse waveform. By acquiring these measurements at multiple locations, both the native waveform shape and cross-terms can be assessed to calculate the gradient input response function and design pre-emphasis.
1.2. Measurable standards for gradients include ramp time <200 us, average gradient strengths 25-40 mT/m over the targeted FOV, and heating below 37° C. for 60% duty cycle.

### 2. Tx/Rx Coil for NuBo

2.1. The Tx/Rx solenoid will be built and connected to newly designed boards for Tx/Rx with MOSFETs at 1 MHz. The coils can be wound from 18 AWG copper wire and placed inside a slotted conductive shielded¹¹³ (the shield is slotted to avoid induced eddy currents on the shield, while still preventing interference from other electronics). After bench measurements of matching and tuning, preliminary scans without a sample can be used to assess ringdown time. Q-damping circuits¹¹⁴ can be implemented if ring-down time is too long. B1+ can then be mapped across the field of view using our automated system.
2.2. Measurable standards for RF include S11>−15 dB, with ring down time less than 50 us and B1+ homogeneity within 5% of relative standard deviation (SD).

### 3. Polarizing Magnet for DBM

3.1. The polarizing magnet can be built from 4.5 mm hollow conductive copper wire with 25-windings per layer and 9 layers. After initial measurements of resistance, inductance and water flow, the system can be matched and tuned to the amplifier (275A Performance Systems, Inc) and tested to full current for assessment of heating and torque. Spatial mapping of the magnetic field can be acquired to assess both magnitude and direction of field at all locations. As with spatial encoding gradients, these can be used to design pre-emphasis for higher fidelity outputs.
3.2. Measurable standards for DBM polarizing magnet include field magnitude between 0.4 and 1.0 T over targeted FOV, ramp times <30 ms, and magnet temperature below 37° C. for 100% duty cycle.

### 4. Gradients for DBM

4.1. Coil manufacture and testing can follow procedures outlined for NuBo gradients; only the winding pattern is significantly different.
4.2. Measurable standards: Also same, i.e., ramp time <200 us and average gradient strengths >40 mT/m over the targeted FOV, heating below 37° C. for 60% duty cycle (our imaging sequences are nowhere near a 60% duty cycle because our sequences require time in the low field imaging phase, and this breaks up the high-field polarization phase).

### 5. Tx/Rx Coil for DBM

5.1. Coil manufacture and testing can follow procedure outlined for NuBo solenoid, as only the winding pattern is significantly different.
5.2. Measurable standards: Also same, i.e., S11 better than −15 dB, with ring down time less than 50 us and B1+ homogeneity within 5% of the standard deviation.

### B. System Developments Applicable to Both NuBo and DBM

### 1. T1w/T2w Sequence Development

1.1 The existing TSE sequence can be the first method tested with each new hardware setup. This can be used to measure SNR across echo trains and slices, both with and without spatial encoding. Gradient refocused spin-warp encoding trajectory can be used for spatial encoding following a polarization period of 6 seconds (polarization accumulates at a rate of T1). Encoding can be set up such that acquisition of central k-space can be either ˜200 ms for a T2w sequence, or ˜10 ms for a T1w sequence. Following initial phantom imaging studies to establish image quality metrics, multi-slice images can be acquired in X healthy volunteers.
1.2. Measurable standards: Resolution in phantoms and in vivo can be better than 1 mm in plane, with slice thickness <3 mm. Temporal resolution for a volume can be <30 seconds.

### 2. Contrast-Enhanced Sequence Development

2.1. Phantom studies using the T1 contrast phantom (water doped with different concentrations of gadobenate dimeglumine) can be improved to achieve the target resolution with less than 2-minute scan time. Imaging contrast in phantoms can also be quantified on both scanners. Following this improvement, the scans can be run on healthy subjects and reviewed by radiologists to confirm in vivo image quality.
2.2. Measurable standards: T1w images can be acquired with 1 mm in plane resolution in under 2 minutes.

### 3. Fat or Water Suppressed Sequence Development

3.1. Fat or water suppression can be achieved using the T1w TSE sequence, manipulating pre-polarization timing or polarity to suppress fat or water. Initial studies can be performed in phantoms of oil and water. The developed sequence can then be tested in healthy volunteers. In vivo images can be assessed by radiologists for image quality.
3.2. Measurable standards: Contrast-to-Noise (CNR)>5 between fat and water in phantom studies and humans.

### 4. DWI Sequence Development

4.1. Diffusion weighted imaging can be accomplished by incorporating ramp up of the polarizing magnet between RF pulses in the first echo times of the train. We can ramp to a through-slice gradient amplitude of >200 mT/m for all slices which can provide high b-value in short echo times. Earlier echo spacings may need to be slightly longer (˜5 ms) to achieve high b-value, but echoes used for spatial encoding can have 2 ms spacing. After initial testing in phantoms to establish image quality metrics, healthy volunteers can be imaged and assessed by radiologists for image quality.
4.2. Measurable standards: In plane resolution can be better than 3 mm in plane with 5 mm slice selection115. As in high field imaging, diffusion weighted images are expected to need somewhat lower spatial resolution due to the SNR loss needed to achieve diffusion contrast.

### 5. Image Reconstruction

5.1. The effect of concomitant fields can be estimated from initial measurements of the magnetic field in polarizing and gradient hardware. These can be incorporated into encoding matrix to properly model phase evolution during spatial encoding. Initial reconstruction algorithms can use GPU-accelerated algebraic reconstruction to provide maximum flexibility so that all hardware characterization can be accurately incorporated into encoding matrix. Future developments can improve reconstruction time and implement denoising and regularization via machine learning.
5.2. Measurable standards: Reconstruction times <2 minutes per volume with SNR improvements >3 dB.

### 6. Standalone Console

6.1. Controller and component boards can be designed to minimize cost and complexity using existing open-source solutions for Tx, Rx, and waveform generator logic. The controller sequencer can be built to both update peripheral boards and cue them to execute. The final implementation can include a sparse user interface that can be unlocked by field engineers to access additional parameters and information.
6.2. Measurable standards: Standalone console coordinates all sequence elements with proper execution.

### 7. Compare Prototypes and Choose Best Scanner

7.1. Data from human and phantom studies can be analyzed for resolution and SNR. Beyond this, we can consider results from contrast studies and image quality ratings.
7.2. Measurable standards: The chosen scanner can meet all predefined metrics for image quality. Beyond this, the system with best SNR, stability.

### Phase 2: Finalize Design and Install Clinical Scanners

### C. Implement Finalized Hardware Design

### 1. Build First Clinical Scanner

1.1. The first clinical scanner can be modified to optimize ergonomics, likely placing the system at an angle so that subjects can sit in a chair during imaging. If DBM is found to be the superior scanner, this is expected to be the only deviation from the prototype. If NuB0 is found to be the best scanner, we can build a breast-improved version, NuB02.0, which provides better polarization, particularly at the chest wall. All manufacturing details of this scanner can be carefully documented and controlled in preparation for FDA filing. QA acceptance can follow the validation protocols outlined for the Phase 1 development scanner.
1.2. Measurable standards: First clinical scanner preferably passes all criteria for performance and safety as outlined previously for development prototype.

### 2. Test First Clinical Scanner

2.1. Final testing of the first clinical scanner, installed at a patient-only room of MRRC, can proceed by imaging 8 subjects in both the prototype and first clinical system. In the unlikely event that image quality is lower than that observed in the prototype, an exact replica of the winning prototype (either DBM or original NuB0 for prone imaging) can be built for installation in clinical sites.
2.2. Measurable standards: First clinical scanner matches performance of development prototype.

### 3. Subsequent Clinical Installations and Commencement of FDA Process

3.1. Careful documentation of all manufacturing, both for first and subsequent clinical scanners, can be gathered for commencement of FDA process. All components can be individually and jointly tested using the criteria for performance and safety outlined for the development prototype. Following finalization of simplified console interface, techs can be trained to run QA as well as contrast-enhanced human studies.
3.2. Measurable standards: Installation of final clinical scanners and commencement of FDA approval process for 510k filing.

### D. System Monitoring for Safety and QA

### 1. Safety Monitoring

1.1. Permanently connected magnetometers can be installed at the edge of each magnet. These, in combination with previously acquired 3D maps, can be used to monitor field stability across time and also to prevent scans that may produce PNS. Notably, the maximum slew rate of our systems (˜0.5 T in 35 ms) is still well below the cardiac stimulation limit116, which itself is set conservatively by about an order of magnitude. B1 can be similarly monitored using a sniffer coil placed outside the FOV and, in combination with previous measurements, can be used to monitor SAR in real-time. These, as well as temperature monitors for all electromagnets can be interfaced to console which can monitor this input to prevent scans that violate safety limits.
1.2. Measurable standards: Scanner automatically detects and prevents scans that violate prescribed dB/dt, SAR, or temperature limits.

### 2. Quality Assurance

2.1. A daily scan protocol can be established to confirm proper system function at the start of each day's operation. These scans can be run on a standard resolution phantom with standard positioning, so that expected data can be easily analyzed. Analyses can confirm that all hardware is within spec including polarizing field strength, gradient shape, RF power, and receiver SNR. EMI functionality can be confirmed via a scan that excludes signal excitation. The console can run this series consecutively and also analyze the data, so no user interaction is required. If service is needed, the system automatically sends results for troubleshooting.
2.2. Measurable standards: scanner performs scan series and analyzes data in an automated manner. Aside from positioning the standardized phantom and beginning the procedure, no further interaction should be required.

### 3. Advanced System Characterization

3.1. Data collected for safety monitoring, along with environmental variables, can be accumulated in tandem with QA scans. This data can be assembled and could be mined to identify patterns between QA image metrics and hardware variables.
3.2. Measurable standards: Contemporaneous data, both image data and monitored variables, from all sites is available and searchable.

### E. Further Improvements to SNR and Speed

### 1. Develop Multichannel Receive for Accelerating Image Acquisitions

1.1. Build separate Tx coil and a multichannel receive coil for winning scanner (winding depends on orientation of polarization fields). Preliminary data has shown the feasibility of decoupling array elements at low field, and this can be refined with either a ladder network or transformer decoupling. Detuning circuits can be built to protect receive chain from Tx power. Scans acquired with multichannel receive can be tested for their ability to shorten scan time via parallel imaging.
1.2. Measurable standards: Acceleration factors of 2 with negligible ghosting artifact can be achieved.

### 2. Improve Images Through Accumulated Data

2.1. Implement reconstructions that better incorporate scanner diagnostic data acquired through QA and system monitoring.
2.2. Measurable standards: Improved SNR or faster reconstruction

### Phase 3: Clinical Testing

### F. Quantify Conspicuity of Known Lesions

1. Acquire Data from Patients with Known Lesions (Biopsy-Confirmed)
1.1. Recruit N=120 patients who have had previous MRI-detectable disease which was later biopsy-confirmed as malignant.
1.2. Measurable standards: 120 patients recruited and imaged within 2 years across 3 sites with mean detectability target score of >4 but acceptable score >3.

### 2. Quantify Detectability

2.1. A team of radiologists can rate detectability of any visible lesions on a 5-point scale. In some cases more than one lesion may be perceived, all lesions can be given score but the most detectable lesion can serve for the primary rating on the 5-point scale. This data can also help train radiologists on the proper interpretation of our images.
2.2. Measurable standards: Average detectability score can be 4 or higher on confirmed lesions.

### G. Quantify Detectability and Suspicion in Lesion-Enriched Population

### 1. Acquire Data in Patients are Going for Biopsy (not Confirmed Cancer).

1.1. 595 patients who had a suspicious finding on previous scan but have not yet received a biopsy can be recruited. Biopsy results from these patients can also be collected.
1.2. Measurable standards: 595 patients can be recruited and imaged within 2 years across 3 sites. We anticipate 20-30% of these patients test positive for cancer. Therefore, this is a screening study with an enriched population.

### 2. Calculate Detectability and Suspicion of Lesions Relative to Mammography

2.1. Identified lesions in each image will be rated for detectability and suspicion on a 5-point scale. With comparison to biopsy results, these will be used to generate comparable 2×2 truth tables, quantify performance, and generate ROC curves.
2.2. Measurable standards: Sensitivity and specificity will be superior to or equal to mammography.

### H. Test Through-Put and Workflow in a Screening Setting (Healthy Subjects)

1. Acquire Screening Breast MRIs on 120 Subjects Recruited from the General Population.
1.1. Subjects can undergo our regular breast MRI protocol and complete a survey on the procedure as well including the study mechanisms (ease of entry/exist, duration, comfort).

### 2. Rate the Image Quality.

2.1. Our radiology team can read all the cases (and flag any that require follow up) as well as rate image quality in terms of contrast, snr, consistency of image quality from case-to-case, and any artifacts (motion).
2.2. Measurable standards: Good quality is consistently achieved and the subject feedback is positive.

The disclosed subject matter can be further illustrated by the following numbered paragraphs:
1. A breast magnetic resonance imaging (MRI) system, preferably compact breast MRI system, suitable for imaging in mixed-use rooms, the breast MRI system containing:
a first component containing an open, field-cycling magnet configured to produce a first non-uniform B₀ magnetic field and a second non-uniform B₀ magnetic field having a field strength different from that of the first non-uniform B₀ magnetic field, and
one or a plurality of second components operably linked to the first component and configured to produce one or a plurality of nonlinear spatial encoding gradients.
2. The breast MRI system of paragraph 1, wherein the open, field-cycling magnet; the spatial encoding gradients; or a combination thereof, are customizable and/or customized to a specific imaging application.
3. The breast MRI system of paragraph 1 or 2, wherein the open, field-cycling magnet and the spatial encoding gradients are customizable and/or customized to a specific imaging application.
4. The breast MRI system of any one of paragraphs 1 to 3, wherein the first non-uniform B₀ magnetic field has a field strength greater than that of the second non-uniform B₀ magnetic field.
5. The breast MRI system of any one of paragraphs 1 to 4, wherein the first non-uniform B₀ magnetic field is configured to generate a slice-selective gradient.
6. The breast MRI system of any one of paragraphs 1 to 5, wherein the magnet is an electromagnet, such as a resistive electromagnet or a superconducting electromagnet.
7. The breast MRI system of any one of paragraphs 1 to 6, wherein the one or the plurality of second components contain: (i) one or more radiofrequency coils and/or parallel receiver coils; (ii) one or more nonlinear gradient coils; or (iii) both (i) and (ii); capable of being configured to produce the one or the plurality of nonlinear spatial encoding gradients.
8. The breast MRI system of any one of paragraphs 1 to 7, wherein the one or plurality of second components contain: (i) one or more radiofrequency coils and/or receiver coils (e.g., parallel receiver coils); (ii) one or more nonlinear DC gradient coils; or (iii) both (i) and (ii); capable of being tailored specifically to the first non-uniform B₀ magnetic field, the second non-uniform B₀ magnetic field, or both.
9. The breast MRI system of any one of paragraphs 1 to 8, wherein the one or plurality of second components contain: (i) one or more radiofrequency coils and/or receiver coils (e.g., parallel receiver coils); (ii) one or more nonlinear DC gradient coils; or (iii) both (i) and (ii); capable of being configured to reconstruct an image of a sample/volume of interest.
10. The breast MRI system of any one of paragraphs 1 to 8, wherein the one or plurality of second components contain one or more radiofrequency coils and/or parallel receiver coils.
11. The breast MRI system of any one of claims 1 to 10, wherein the one or plurality of second components contain one or more radiofrequency coils and/or receiver coils (e.g., parallel receiver coils), wherein the one or more radiofrequency coils have a non-planar configuration, a non-horizontal configuration, or both.
12. The breast MRI system of any one of paragraphs 1 to 9, wherein the plurality of nonlinear spatial encoding gradients spans a three-dimensional volume space, are sequentially generated in an imaging region, and/or separately generated in an imaging region.
13. The breast MRI system of any one of paragraphs 1 to 12, wherein:
the first non-uniform B₀ magnetic field polarizes spins in a sample/volume of interest, and/or
the second non-uniform B₀ magnetic field is applied in an imaging phase for the sample/volume of interest.
14. The breast MRI system of any one of paragraphs 1 to 13, operably linked to a computing device that controls the operation of the open, field-cycling magnet; the one or the plurality of second components; the one or more radiofrequency coils; the one or more receiver coils (e.g., parallel receiver coils); the one or more nonlinear DC gradient coils; or a combination thereof, preferably wherein the computing device contains tablet computers, servers, desktop computers, laptop computers, mainframes, or a combination thereof.
15. A method of imaging a breast anatomy of interest using the breast MRI system of any one of paragraphs 1 to 14, the method involving:
field cycling between the first non-uniform B₀ magnetic field and the second non-uniform B₀ magnetic field.
16. The method of claim 15, wherein the first non-uniform B₀ magnetic field is applied to the sample/volume of interest prior to applying the second non-uniform B₀ magnetic field to the sample/volume of interest.
17. The method of paragraph 15 or 16, wherein field cycling between the first non-uniform B₀ magnetic field and the second non-uniform B₀ magnetic field is achieved by controlling/altering a current supplied to the magnet.
18. The method of any one of paragraphs 15 to 17, wherein MRI images are reconstructed from (parallel or single-channel) received magnetic resonance signals from RF coils using algebraic reconstruction.
19. The method of paragraph 19, wherein image reconstruction does not use standard Fourier-based image reconstruction.

It will be appreciated that variations in intensities and alternatives in elements of the components shown will be apparent to those skilled in the art and are within the scope of disclosed forms. All parts or amounts, unless otherwise specified, are by weight. Use of the term “about” is intended to describe values either above or below the stated value in a range of approx. +/−10%. Examples of values within this range are +/−1%, +/−2%, +/−3%, +/−4%, +/−5%, +/−6%, +/−7%, +/−8%, +/−9%, and +/−10%.

### REFERENCES

1 Mehring, M., Höfer, P. & Grupp, A. Bloch-Siegert shift, Rabi oscillation, and spinor behavior in pulsed electron-nuclear double-resonance experiments. Phys Rev A Gen Phys 33, 3523-3526 (1986).
2 Sacolick, L. I., Wiesinger, F., Hancu, I. & Vogel, M. W. B1 mapping by Bloch-Siegert shift. Magnetic resonance in medicine: official journal of the Society of Magnetic Resonance in Medicine/Society of Magnetic Resonance in Medicine 63, 1315-1322 (2010).
3 Lång, K. et al. Artificial intelligence-supported screen reading versus standard double reading in the Mammography Screening with Artificial Intelligence trial (MASAI): a clinical safety analysis of a randomised, controlled, non-inferiority, single-blinded, screening accuracy study. The Lancet Oncology 24, 936-944 (2023).
4 Oeffinger, K. C. et al. Breast cancer screening for women at average risk: 2015 Guideline update from the American cancer society. JAMA-Journal of the American Medical Association 314, 1599-1614 (2015).
5 Siu, A. L. Screening for breast cancer: U.S. Preventive services task force recommendation statement. Annals of Internal Medicine 164, 279-296 (2016).
6 Hollingsworth, A. B. Redefining the sensitivity of screening mammography: A review. The American Journal of Surgery 218, 411-418 (2019).
7 Mainiero, M. B. et al. ACR Appropriateness Criteria® Breast Cancer Screening. Journal of the American College of Radiology 14, S383-S390 (2017).
8 Mann, R. M., Cho, N. & Moy, L. Breast MRI: State of the art. Radiology 292, 520-536 (2019).
9 Mann, R. M., Kuhl, C. K. & Moy, L. Contrast-enhanced MRI for breast cancer screening. Journal of Magnetic Resonance Imaging 50, 377-390 (2019).
10 Kuhl, C. K. Abbreviated breast MRI for screening women with dense breast: the EA1141 trial. The British journal of radiology 91, 20170441 (2018).
11 Bakker, M. F. et al. Supplemental MRI screening for women with extremely dense breast tissue. New England Journal of Medicine 381, 2091-2102 (2019).
12 Vourtsis, A. & Berg, W. A. Breast density implications and supplemental screening. European Radiology 29, 1762-1777 (2019).
13 Mann, R. M. et al. Breast cancer screening in women with extremely dense breasts recommendations of the European Society of Breast Imaging (EUSOBI). European Radiology 32, 4036-4045 (2022).
14 Shao, H. et al. Comparison of the diagnostic efficiency for breast cancer in Chinese women using mammography, ultrasound, MRI, and different combinations of these imaging modalities. Journal of X-Ray Science & Technology 21, 283-292 (2013).
15 Pickles, M. D. et al. Comparison of 3.0 T magnetic resonance imaging and X-ray mammography in the measurement of ductal carcinoma in situ: A comparison with histopathology. European Journal of Radiology 84, 603-610 (2015).
16 Lee, T. C., Reyna, C., Shaughnessy, E. & Lewis, J. D. Screening of populations at high risk for breast cancer. Journal of Surgical Oncology 120, 820-830 (2019).
17 Sheth, D. & Giger, M. L. Artificial intelligence in the interpretation of breast cancer on MRI. Journal of Magnetic Resonance Imaging 51, 1310-1324 (2020).
18 Mckinney, S. M. et al. International evaluation of an AI system for breast cancer screening. Nature 577, 89-94 (2020).
19 Hosny, A., Parmar, C., Quackenbush, J., Schwartz, L. H. & Aerts, H. J. W. L. Artificial intelligence in radiology. Nature Reviews Cancer 18, 500-510 (2018).
20 Mckinney, S. M. et al. International evaluation of an AI system for breast cancer screening. Nature 577, 89-94 (2020).
21 Sharma, P., Suehling, M., Flohr, T. & Comaniciu, D. Artificial Intelligence in Diagnostic Imaging: Status Quo, Challenges, and Future Opportunities. Journal of Thoracic Imaging 35, S11-S16 (2020).
22 Hancu, I. et al. Flexible, 31-Channel breast coil for enhanced parallel imaging performance at 3 T. Magnetic Resonance in Medicine 75, 897-905 (2016).
23 Hancu, I. et al. Field shaping arrays: a means to address shading in high field breast MRI. Journal of magnetic resonance imaging: JMRI 36, 865-872 (2012).
24 Sodickson, D. K. & Manning, W. J. Simultaneous acquisition of spatial harmonics (SMASH): fast imaging with radiofrequency coil arrays. Magnetic resonance in medicine: official journal of the Society of Magnetic Resonance in Medicine/Society of Magnetic Resonance in Medicine 38, 591-603 (1997).
25 Pruessmann, K. P., Weiger, M., Scheidegger, M. B. & Boesiger, P. SENSE: sensitivity encoding for fast MRI. Magnetic resonance in medicine: official journal of the Society of Magnetic Resonance in Medicine/Society of Magnetic Resonance in Medicine 42, 952-962 (1999).
26 Anand, S. S., JP; Wald, L L, Witzel, T.; A low-cost (<$500 USD) FPGA-based console capable of real-time control. Proc Intl Soc Magn Reson Med Paris (2018).
27 IEEE Standard for Verilog Hardware Description Language. IEEE Std 1364-2005 (Revision of IEEE Std 1364-2001), 1-590 (2006).
28 Van Norman, G. A. Drugs, Devices, and the FDA: Part 2: An Overview of Approval Processes: FDA Approval of Medical Devices. JACC: Basic to Translational Science 1, 277-287 (2016).
29 Society, A. C. American Cancer Society Recommendations for the early detection of breast cancer. online on the cancer.org website.
30 Comstock, C. E. et al. Comparison of Abbreviated Breast MRI vs Digital Breast Tomosynthesis for Breast Cancer Detection among Women with Dense Breasts Undergoing Screening. JAMA-Journal of the American Medical Association 323, 746-756 (2020).
31 Haas, J. S. et al. Disparities in the use of screening magnetic resonance imaging of the breast in community practice by race, ethnicity, and socioeconomic status. Cancer 122, 611-617 (2016).
32 Wernli, K. J. et al. Surveillance Breast MRI and Mammography: Comparison in Women with a Personal History of Breast Cancer. Radiology 292, 311-318 (2019).
33 Onega, T. et al. Travel Burden to Breast MRI and Utilization: Are Risk and Sociodemographic Related? Journal of the American College of Radiology 13, 611-619 (2016).
34 Moore, S. G., Shenoy, P. J., Fanucchi, L., Tumeh, J. W. & Flowers, C. R. Cost-effectiveness of MRI compared to mammography for breast cancer screening in a high risk population. BMC Health Services Research 9, 9 (2009).
35 Rothberg, J. Hyperfine: website hyperfine.io, (2020).
36 Sheth, K. N. et al. Assessment of Brain Injury Using Portable, Low-Field Magnetic Resonance Imaging at the Bedside of Critically Ill Patients. JAMA Neurology (2020).
37 Wald, L. Halbach Magnet: website nmr.mgh.harvard.edu, (2020).
38 Snyder, A. L., Corum, C. A., Moeller, S., Powell, N. J. & Garwood, M. MRI by steering resonance through space. Magnetic resonance in medicine: official journal of the Society of Magnetic Resonance in Medicine/Society of Magnetic Resonance in Medicine 72, 49-58 (2014).
39 Kriege, M. et al. Efficacy of MRI and Mammography for Breast-Cancer Screening in Women with a Familial or Genetic Predisposition. New England Journal of Medicine 351, 427-437 (2004).
40 Rebolj, M., Assi, V., Brentnall, A., Parmar, D. & Duffy, S. W. Addition of ultrasound to mammography in the case of dense breast tissue: systematic review and meta-analysis. British Journal of Cancer 118, 1559-1570 (2018).
41 Wernli, K. J. et al. Patterns of breast magnetic resonance imaging use in community practice. JAMA Intern Med 174, 125-132 (2014).
42 Statistics, C. N. C. f. H. Health United States 2020-2021. Mammography (2023).
43 Tollens, F. et al. Economic potential of abbreviated breast MRI for screening women with dense breast tissue for breast cancer. European Radiology 32, 7409-7419 (2022).
44 Geuzinge, H. A. et al. Cost-effectiveness of Breast Cancer Screening With Magnetic Resonance Imaging for Women at Familial Risk. JAMA Oncology 6, 1381-1389 (2020).
45 Martincich, L. et al. Correlations between diffusion-weighted imaging and breast cancer biomarkers. European Radiology 22, 1519-1528 (2012). 46 Lo Gullo, R. et al. A survey by the European Society of Breast Imaging on the implementation of breast diffusion-weighted imaging in clinical practice. European Radiology 32, 6588-6597 (2022).
47 Bickelhaupt, S. et al. Prediction of malignancy by a radiomic signature from contrast agent-free diffusion MRI in suspicious breast lesions found on screening mammography. Journal of Magnetic Resonance Imaging 46, 604-616 (2017).
48 Saccenti, L. et al. Combining b2500 diffusion-weighted imaging with BI-RADS improves the specificity of breast MRI. Diagn Interv Imaging 104, 410-418 (2023).
49 Pinker, K. et al. Diffusion-Weighted Imaging With Apparent Diffusion Coefficient Mapping for Breast Cancer Detection as a Stand-Alone Parameter: Comparison With Dynamic Contrast-Enhanced and Multiparametric Magnetic Resonance Imaging. Invest Radiol 53, 587-595 (2018).
50 Baltzer, P. A. T. et al. Sensitivity and specificity of unenhanced MR mammography (DWI combined with T2-weighted TSE imaging, ueMRM) for the differentiation of mass lesions. European Radiology 20, 1101-1110 (2010).
51 (ISO), I. O. f. S. Medical devices—Quality management systems—Requirements for regulatory purposes [Internet]. Geneva, C H.; (2016).
52 AAMI, A. Guidance on the use of agile practices in the development of medical device software. Association for the Advancement of Medical Instrumentation 21, 22-78 (2012).
53 (IEC), I. E. C. IEC 62304 Medical device software—Software life cycle processes [Internet]. Geneva, C H.; (2006).
54 Messiry, M. E. & Mohamed, A. Innovative technique in sheet forming of feminine personal protective armor by combined cyclic stretching and deep drawing method. Journal of Engineered Fibers and Fabrics 17, 15589250221144009 (2022).
55 Amrein, P., Jia, F., Zaitsev, M. & Littin, S. CoilGen: Open-source MR coil layout generator. Magnetic Resonance in Medicine 88, 1465-1479 (2022).
56 Evetts, N. & Conradi, M. S. Low-cost gradient amplifiers for small MRI systems. Journal of Magnetic Resonance 335, 107127 (2022).
57 Wolfman, N., Moran, R., Moran, P. & Karstaedt, N. Simultaneous MR imaging of both breasts using a dedicated receiver coil. Radiology 155, 241-243 (1985).
58 Sun, L., Olsen, J. O. & Robitaille, P. M. L. Design and optimization of a breast coil for magnetic resonance imaging. Magnetic resonance imaging 11, 73-80 (1993).
59 Fiedler, T. M., Ladd, M. E. & Bitz, A. K. RF safety assessment of a bilateral four-channel transmit/receive 7 Tesla breast coil: SAR versus tissue temperature limits. Medical physics 44, 143-157 (2017).
60 Cui, J. et al. A switched-mode breast coil for 7 T MRI using forced-current excitation. IEEE transactions on biomedical engineering 62, 1777-1783 (2015).
61 Roemer, P. B., Edelstein, W. A., Hayes, C. E., Souza, S. P. & Mueller, O. M. The NMR phased array. Magnetic resonance in medicine 16, 192-225 (1990).
62 Nnewihe, A. N. et al. Custom-fitted 16-channel bilateral breast coil for bidirectional parallel imaging. Magnetic Resonance in Medicine 66, 281-289 (2011).
63 Kaggie, J. D. et al. A 3 T sodium and proton composite array breast coil. Magnetic resonance in medicine 71, 2231-2242 (2014).
64 Ianniello, C., Madelin, G., Moy, L. & Brown, R. A dual-tuned multichannel bilateral RF coil for 1H/23Na breast MRI at 7 T. Magnetic resonance in medicine 82, 1566-1575 (2019).
65 Hancu, I. et al. Flexible, 31-channel breast coil for enhanced parallel imaging performance at 3 T. Magnetic resonance in medicine 75, 897-905 (2016).
66 Vincent, J. et al. in ISMRM.
67 Obermann, M. et al. Panoramic Magnetic Resonance Imaging of the Breast With a Wearable Coil Vest. Investigative Radiology, 10.1097 (2023).
68 Komu, M. & Kormano, M. Breast coil design for low-field MRI. Magnetic resonance in medicine 27, 165-170 (1992).
69 Blasiak, B., Volotovskyy, V., Tyson, R., Sharp, J. & Tomanek, B. An RF breast coil for 0.2 T MRI. Concepts in Magnetic Resonance Part B: Magnetic Resonance Engineering 46, 3-7 (2016).
70 Davies, G. R., Broche, L., Gagliardi, T., Lurie, D. J. & Ross, P. J. in 27th Annual Meeting, International Society for Magnetic Resonance in Medicine.
71 Neha Koonjoo, T. P. P. H., Susu Yan, Matthew S Rosen, and Thomas R Bortfeld. Low field Breast and Chest Wall Imaging for MR guided Proton Therapy 3895. Proc. Intl. Soc. Mag. Reson. Med. 31 (2023).
72 Cooley, C. Z. et al. Two-dimensional imaging in a lightweight portable MRI scanner without gradient coils. Magnetic resonance in medicine 73, 872-883 (2015).
73 de Vos, B. et al. Design, characterisation and performance of an improved portable and sustainable low-field MRI system. Frontiers in Physics, 413 (2021).
74 Barberi, E. A., Gati, J. S., Rutt, B. K. & Menon, R. S. A transmit-only/receive-only (TORO) RF system for high-field MRI/MRS applications. Magnetic Resonance in Medicine: An Official Journal of the International Society for Magnetic Resonance in Medicine 43, 284-289 (2000).
75 Nacher, P. J., Kumaragamage, S., Tastevin, G. & Bidinosti, C. P. A fast MOSFET rf switch for low-field NMR and MRI. Journal of Magnetic Resonance 310, 106638 (2020).
76 Mao, W., Smith, M. B. & Collins, C. M. Exploring the limits of RF shimming for high-field MRI of the human head. Magnetic Resonance in Medicine: An Official Journal of the International Society for Magnetic Resonance in Medicine 56, 918-922 (2006).
77 Tam, L. K. et al. Pseudo-random center placement O-space imaging for improved incoherence compressed sensing parallel MRI. Magnetic resonance in medicine 73, 2212-2224 (2015).
78 Tam, L. K., Stockmann, J. P., Galiana, G. & Constable, R. T. Null space imaging: nonlinear magnetic encoding fields designed complementary to receiver coil sensitivities for improved acceleration in parallel imaging. Magnetic resonance in medicine 68, 1166-1175 (2012).
79 Tam, L. K., Stockmann, J. P., Galiana, G., Peters, D. C. & Constable, R. T. 3D Null Space Imaging: Nonlinear magnetic encoding gradients designed complementary to 3D coil sensitivity profiles for rapid volume imaging. Magn. Reson. Med 51, 172 (2004).
80 Stockmann, J. P., Ciris, P. A., Galiana, G., Tam, L. & Constable, R. T. O-space imaging: highly efficient parallel imaging using second-order nonlinear fields as encoding gradients with no phase encoding. Magnetic resonance in medicine 64, 447-456 (2010).
81 Stockmann, J. P. et al. In vivo O-Space imaging with a dedicated 12 cm Z2 insert coil on a human 3 T scanner using phase map calibration. Magnetic resonance in medicine 69, 444-455 (2013).
82 Salmon, C. E. G., Vidoto, E. L. G., Martins, M. J. & Tannús, A. Optimization of saddle coils for magnetic resonance imaging. Brazilian Journal of Physics 36, 4-8 (2006).
83 Mann, R. M., Kuhl, C. K., Kinkel, K. & Boetes, C. Breast MRI: guidelines from the European Society of Breast Imaging. Eur Radiol 18, 1307-1318 (2008).
84 Pediconi, F. et al. Contrast-enhanced MR mammography: improved lesion detection and differentiation with gadobenate dimeglumine. AJR Am J Roentgenol 191, 1339-1346 (2008).
85 Carbonaro, L. A. et al. Breast MRI using a high-relaxivity contrast agent: an overview. AJR Am J Roentgenol 196, 942-955 (2011).
86 Kuhl, C. K. The Changing World of Breast Cancer: A Radiologist's Perspective. Invest Radiol 50, 615-628 (2015).
87 Carbonaro, L. A. et al. Breast MRI Using a High-Relaxivity Contrast Agent: An Overview. American Journal of Roentgenology 196, 942-955 (2011).
88 Luciani, M. L., Telesca, M., Kirchin, M. A. & Pediconi, F. Clinical utility of gadobenate dimeglumine in contrast-enhanced MRI of the breast: a review. Research and Reports in Nuclear Medicine 5, 33-40 (2015).
89 Hori, M., Hagiwara, A., Goto, M., Wada, A. & Aoki, S. Low-Field Magnetic Resonance Imaging: Its History and Renaissance. Invest Radiol 56, 669-679 (2021).
90 Bickelhaupt, S. et al. Fast and Noninvasive Characterization of Suspicious Lesions Detected at Breast Cancer X-Ray Screening: Capability of Diffusion-weighted MR Imaging with MIPs. Radiology 278, 689-697 (2016).
91 Stadlbauer, A. et al. Diffusion-weighted MR imaging with background body signal suppression (DWIBS) for the diagnosis of malignant and benign breast lesions. European Radiology 19, 2349-2356 (2009).
92 Stockmann, J. P. & Constable, R. T. in Proc. Intl. Soc. Mag. Reson. Med. 2857.
93 Stockmann, J. P. & Constable, R. T. in Proc. Intl. Soc. Mag. Reson. Med. 3078.
94 Stockmann, J. P. & Constable, R. T. in Proc. Joint Annual Meeting ISMRM-ESMRMB. 549.
95 Galiana, G. & Constable, R. T. Single echo MRI. PloS one 9, e86008 (2014).
96 Galiana, G., Peters, D., Tam, L. & Constable, R. T. Multiecho acquisition of O-space data. Magnetic resonance in medicine: official journal of the Society of Magnetic Resonance in Medicine/Society of Magnetic Resonance in Medicine 72, 1648-1657 (2014).
97 Galiana, G., Stockmann, J. P., Tam, L. & Constable, R. T. Spin dephasing under nonlinear gradients: Implications for imaging and field mapping. Magnetic Resonance in Medicine 67, 1120-1126 (2012).
98 Galiana, G. et al. The Role of Nonlinear Gradients in Parallel Imaging: A k-Space Based Analysis. Concepts in magnetic resonance. Part A, Bridging education and research 40a, 253-267 (2012).
99 Hoque Bhuiyan, E., Dewdney, A., Weinreb, J. & Galiana, G. Feasibility of diffusion weighting with a local inside-out nonlinear gradient coil for prostate MRI. Medical physics 48, 5804-5818 (2021).
100 Fettahoglu, G. Z., Z; Elsaid, N; Dewdney, A; Constable, R T; Galiana, G.; Eddy-current characterization and pre-emphasis on a compact inside-out nonlinear gradient. Proc. of ISMRM, 4431 (2023).
101 Andersson, J. L. R. & Sotiropoulos, S. N. An integrated approach to correction for off-resonance effects and subject movement in diffusion MR imaging. NeuroImage 125, 1063-1078 (2016).
102 Volegov, P. L., Mosher, J. C., Espy, M. A. & Kraus, R. H. On concomitant gradients in low-field MRI. Journal of Magnetic Resonance 175, 103-113 (2005).
103 Mango, V. L. et al. Abbreviated protocol for breast MRI: Are multiple sequences needed for cancer detection? European Journal of Radiology 84, 65-70 (2015).
104 Kuhl, C. K. et al. Abbreviated breast magnetic resonance imaging (MRI): first postcontrast subtracted images and maximum-intensity projection—a novel approach to breast cancer screening with MRI. Journal of clinical oncology: official journal of the American Society of Clinical Oncology 32, 2304-2310 (2014).
105 Takeda, K. A highly integrated FPGA-based nuclear magnetic resonance spectrometer. Rev Sci Instrum 78, 033103 (2007).
106 Feng, K., Hollingsworth, N. A., McDougall, M. P. & Wright, S. M. A 64-Channel Transmitter for Investigating Parallel Transmit MRI. IEEE Transactions on Biomedical Engineering 59, 2152-2160 (2012).
107 Stang, P., Conolly, S., Santos, J., Pauly, J. & Scott, G. Medusa: a scalable MR console using USB. IEEE transactions on medical imaging 31, 370-379 (2011).
108 Dalal, I. L., Kirpalani, A. L. & Fontaine, F. L. A low-cost scalable multichannel digital receiver for magnetic resonance imaging. Conf Proc IEEE Eng Med Biol Soc 2006, 1897-1900 (2006).
109 Jevtic, J. (Google Patents, 2006).
110 Nabeshima, T., Takahashi, T., Matsunaga, Y., Yamamoto, E. & Katakura, K. (Google Patents, 1996).
111 Romeo, V. et al. Preliminary Results of a Simplified Breast MRI Protocol to Characterize Breast Lesions: Comparison with a Full Diagnostic Protocol and a Review of the Current Literature. Academic radiology 24, 1387-1394 (2017).
112 Pötsch, N., Vatteroni, G., Clauser, P., Helbich, T. H. & Baltzer, P. A. T. Contrast-enhanced Mammography versus Contrast-enhanced Breast MRI: A Systematic Review and Meta-Analysis. Radiology 305, 94-103 (2022).
113 Alon, L. et al. Transverse slot antennas for high field MRI. Magnetic Resonance in Medicine 80, 1233-1242 (2018).
114 Peshkovsky, A. S., Forguez, J., Cerioni, L. & Pusiol, D. J. RF probe recovery time reduction with a novel active ringing suppression circuit. Journal of Magnetic Resonance 177, 67-73 (2005).
115 Peng, S. et al. High-Resolution DWI with Simultaneous Multi-Slice Readout-Segmented Echo Planar Imaging for the Evaluation of Malignant and Benign Breast Lesions. Diagnostics 11, 2273 (2021).
116 Klein, V., Davids, M., Schad, L. R., Wald, L. L. & Guérin, B. Investigating cardiac stimulation limits of MRI gradient coils using electromagnetic and electrophysiological simulations in human and canine body models. Magnetic resonance in medicine 85, 1047-1061 (2021).

Those skilled in the art will recognize, or be able to ascertain using no more than routine experimentation, many equivalents to the specific embodiments of the invention described herein. Such equivalents are intended to be encompassed by the following claims.

We claim:

1. A breast magnetic resonance imaging (MRI) system, preferably compact breast MRI system, suitable for imaging in mixed-use rooms, the breast MRI system comprising:
a first component comprising an open, field-cycling magnet configured to produce a first non-uniform B₀ magnetic field and a second non-uniform B₀ magnetic field having a field strength different from that of the first non-uniform B₀ magnetic field, and
one or a plurality of second components operably linked to the first component and configured to produce one or a plurality of nonlinear spatial encoding gradients.

2. The breast MRI system of claim 1, wherein the open, field-cycling magnet; the spatial encoding gradients; or a combination thereof, are customizable and/or customized to a specific imaging application.

3. The breast MRI system of claim 1, wherein the open, field-cycling magnet and the spatial encoding gradients are customizable and/or customized to a specific imaging application.

4. The breast MRI system of claim 1, wherein the first non-uniform B₀ magnetic field has a field strength greater than that of the second non-uniform B₀ magnetic field.

5. The breast MRI system of claim 1, wherein the first non-uniform B₀ magnetic field is configured to generate a slice-selective gradient.

6. The breast MRI system of claim 1, wherein the magnet is an electromagnet, such as a resistive electromagnet or a superconducting electromagnet.

7. The breast MRI system of claim 1, wherein the one or the plurality of second components comprise: (i) one or more radiofrequency coils and/or parallel receiver coils; (ii) one or more nonlinear gradient coils; or (iii) both (i) and (ii); capable of being configured to produce the one or the plurality of nonlinear spatial encoding gradients.

8. The breast MRI system of claim 1, wherein the one or plurality of second components comprise: (i) one or more radiofrequency coils and/or receiver coils (e.g., parallel receiver coils); (ii) one or more nonlinear DC gradient coils; or (iii) both (i) and (ii); capable of being tailored specifically to the first non-uniform B₀ magnetic field, the second non-uniform B₀ magnetic field, or both.

9. The breast MRI system of claim 1, wherein the one or plurality of second components comprise: (i) one or more radiofrequency coils and/or receiver coils (e.g., parallel receiver coils); (ii) one or more nonlinear DC gradient coils; or (iii) both (i) and (ii); capable of being configured to reconstruct an image of a sample/volume of interest.

10. The breast MRI system of claim 1, wherein the one or plurality of second components comprise one or more radiofrequency coils and/or parallel receiver coils.

11. The breast MRI system of claim 1, wherein the one or plurality of second components comprise one or more radiofrequency coils and/or receiver coils, wherein the one or more radiofrequency coils have a non-planar configuration, a non-horizontal configuration, or both.

12. The breast MRI system of claim 1, wherein the plurality of nonlinear spatial encoding gradients spans a three-dimensional volume space, are sequentially generated in an imaging region, and/or separately generated in an imaging region.

13. The breast MRI system of claim 1, wherein:
the first non-uniform B₀ magnetic field polarizes spins in a sample/volume of interest, and/or
the second non-uniform B₀ magnetic field is applied in an imaging phase for the sample/volume of interest.

14. The breast MRI system of claim 1, operably linked to a computing device that controls the operation of the open, field-cycling magnet; the one or the plurality of second components; the one or more radiofrequency coils;
the one or more receiver coils; the one or more nonlinear DC gradient coils;
or a combination thereof.

15. A method of imaging a breast anatomy of interest using the breast MRI system of claim 1, the method comprising:
field cycling between the first non-uniform B₀ magnetic field and the second non-uniform B₀ magnetic field.

16. The method of claim 15, wherein the first non-uniform B₀ magnetic field is applied to the sample/volume of interest prior to applying the second non-uniform B₀ magnetic field to the sample/volume of interest.

17. The method of claim 15, wherein field cycling between the first non-uniform B₀ magnetic field and the second non-uniform B₀ magnetic field is achieved by controlling/altering a current supplied to the magnet.

18. The method of claim 15, wherein MRI images are reconstructed from received magnetic resonance signals from RF coils using algebraic reconstruction.

19. The method of claim 19, wherein image reconstruction does not use standard Fourier-based image reconstruction.
